(19) United States
(12) Patent Application Publication
(10) Pub. No.: US 2026/0069703 A1
(43) Pub. Date: Mar. 12, 2026

(54) RECOMBINANT POLYPEPTIDES AND METHODS FOR USING RECOMBINANT POLYPEPTIDES

(71) Applicant: The Johns Hopkins University, Baltimore, MD (US)

(72) Inventors: Zachary J. Bernstein, Baltimore, MD (US); Taylor R. Gierke, New Haven, CT (US); Kris Dammen-Brower, Baltimore, MD (US); Jamie Spangler, Baltimore, MD (US); Kevin J. Yarema, Woodstock, MD (US)

(21) Appl. No.: 19/322,032

(22) Filed: Sep. 8, 2025

Related U.S. Application Data
(60) Provisional application No. 63/692,276, filed on Sep. 9, 2024.

(51) Int. Cl. A61K 47/68; C07K 14/735

(52) U.S. Cl. CPC A61K 47/68031; A61K 47/6855; C07K 14/70535

(57) ABSTRACT

This document provides methods and materials for producing polypeptide conjugates. For example, recombinant polypeptides (e.g., recombinant fragment crystallizable region (Fc) polypeptides) that include one or more engineered N-linked glycosylation sites are provided as well as methods for using such recombinant polypeptides to generate polypeptide conjugates. In some cases, antibodies including a recombinant Fc polypeptide provided herein can be contacted with one or more carbohydrates each containing a first functional group (e.g., a first bioorthogonal functional group) to produce functionalized polypeptides (e.g., functionalized antibodies) which can be contacted with one or more molecules each including a second functional group (e.g., a second bioorthogonal functional group) such that the first functional group and the second functional group are conjugated thus forming a polypeptide conjugate (e.g., an antibody conjugate) including the functionalized recombinant polypeptides and the molecule(s).

### CROSS-REFERENCE TO RELATED APPLICATIONS

This application claims the benefit of U.S. Patent Application Ser. No. 63/692,276, filed on Sep. 9, 2024. The disclosure of the prior application is considered part of, and is incorporated by reference in, the disclosure of this application.

### SEQUENCE LISTING

This application contains a Sequence Listing that has been submitted electronically as an XML file named “44807-0496001_SL.xml.” The XML file, created on Aug. 18, 2025, is 12,307 bytes in size. The material in the XML file is hereby incorporated by reference in its entirety.

### STATEMENT REGARDING FEDERAL FUNDING

This invention was made with government support under grant number CA249381, awarded by the National Institutes of Health. The government has certain rights in the invention.

### TECHNICAL FIELD

This document relates to methods and materials for producing polypeptide conjugates. For example, this document provides recombinant polypeptides (e.g., recombinant fragment crystallizable region (Fc) polypeptides) that include one or more engineered N-linked glycosylation sites. This document also provides methods for using the recombinant polypeptides provided herein to generate polypeptide conjugates. For example, polypeptides including one or more recombinant polypeptides provided herein (e.g., antibodies including a recombinant Fc polypeptide that comprises, consists essentially of, or consists of an amino acid sequence set forth in any one of SEQ ID NOs:3-8) can be contacted (e.g., in cell culture during cellular translation) with one or more carbohydrates each containing a first functional group (e.g., a first bioorthogonal functional group) to produce functionalized recombinant polypeptides (e.g., functionalized antibodies) which can be contacted with one or more molecules each including a second functional group (e.g., a second bioorthogonal functional group) such that the first functional group and the second functional group are conjugated thus forming a polypeptide conjugate (e.g., an antibody conjugate) including the functionalized recombinant polypeptides and the molecule(s).

### BACKGROUND

Monoclonal antibodies comprise a growing field of biological agents with various applications in basic research and medicine including imaging, targeted gene therapy, surface receptor modulation, and cytotoxic drug delivery (Castelli et al., Pharmacol Res Perspect 7:e00535 (2019) L. Liu, Protein Cell 9:15-32 (2018); Paul et al., Nat Rev Cancer 24:399-426 (2024) Wei et al., Mol. Pharmaceutics 19:3453-3455 (2022); and Wu et al., Med 4:69-74 (2023)). Many antibody applications rely on conjugating secondary agents that leverage the unique specificity of antibodies to target the activities of the conjugated agents. However, current standard approaches for drug conjugation (such as amine or thiol chemistry) are often destructive to antibody structure/stability, lead to non-specific conjugation of the drug throughout the entire antibody, require extensive modification of the antibody sequence, or require multi-component reactions (Matsuda et al., Expert Opinion on Biological Therapy 21:963-975 (2021); and Yamada et al., ChemBioChem 20:2729-2737 (2019)). As a result, these conjugation strategies frequently risk adverse effects on antibody yield, stability, and potentially antigen binding.

N-linked glycosylation is a natural occurring co-translational modification involving the attachment of oligosaccharides to the amide nitrogen of asparagine located within a specific consensus sequence (Asn-X-Ser/Thr, where X is any amino acid except proline) (Hart et al., Journal of Biological Chemistry 254:9747-9753 (1979)). This process is near ubiquitous for cell surface and secreted proteins, including antibodies. Metabolic glycoengineering technologies take advantage of this natural process to introduce modified, non-natural sugars into endogenous glycans, allowing for chemically selective reactions (Dammen-Brower et al., Front Chem 10:863118 (2022)). A pitfall for this approach, however, is that many monosaccharide analogs used in metabolic glycoengineering are hampered by poor cellular uptake.

### SUMMARY

This document provides methods and materials for producing polypeptide conjugates (e.g., antibody conjugates). For example, this document provides recombinant polypeptides (e.g., recombinant fragment crystallizable region (Fc) polypeptides) having (e.g., engineered to have) one or more (e.g., one, two, three, four, or more) N-linked glycosylation sites. Recombinant polypeptides provided herein (e.g., recombinant Fc polypeptides) having one or more engineered N-linked glycosylation sites can also be referred to as recombinant polypeptide glycovariants (e.g., recombinant Fc glycovariants).

This document also provides methods for using the recombinant polypeptides provided herein (e.g., recombinant Fc polypeptides having (e.g., engineered to have) one or more (e.g., one, two, three, four, or more) N-linked glycosylation sites) to generate polypeptide conjugates. For example, polypeptides including one or more recombinant polypeptides provided herein (e.g., antibodies including a recombinant Fc polypeptide provided herein) can be contacted (e.g., in cell culture during cellular translation) with one or more carbohydrates each containing a first functional group to produce functionalized recombinant polypeptides (e.g., functionalized antibodies). Recombinant polypeptides provided herein (e.g., recombinant Fc polypeptides that comprise, consist essentially of, or consist of an amino acid sequence set forth in any one of SEQ ID NOs:3-8) that have been functionalized as described herein (e.g., by contacting a recombinant polypeptide provided herein with one or more carbohydrates each containing a functional group (also referred to functionalized carbohydrates) can also be referred to as functionalized recombinant polypeptides (e.g., functionalized recombinant Fc polypeptides). In some cases, functionalized recombinant polypeptides (e.g., functionalized antibodies) functionalized with a first functional group can be contacted with one or more molecules each including a second functional group that is reactive with the first functional group such that the first functional group and the second functional group can ligate the functionalized recombinant polypeptides with the molecule(s) to form polypeptide conjugates (e.g., antibody conjugates).

As demonstrated herein, recombinant Fc polypeptides containing (e.g., engineered to contain) one or more engineered N-linked glycosylation sites can be incorporated into antibodies and can be used as conjugation sites. For example, a recombinant Fc polypeptide provided herein can be contacted with a carbohydrate containing a functional group (e.g., an azide-functionalized ManNAc analog) such that the functional group is conjugated to the recombinant Fc polypeptide (e.g., to generate functionalized recombinant Fc polypeptides). In some cases, antibodies containing a recombinant Fc polypeptide provided herein can be produced by cells cultured with a carbohydrate containing a functional group (e.g., an azide-functionalized ManNAc analog) such that the functional group is conjugated to the recombinant Fc polypeptide (e.g., to generate functionalized antibodies).

Also as demonstrated herein, recombinant Fc polypeptides containing (e.g., engineered to contain) one or more engineered N-linked glycosylation sites can be used in a glycoengineering approach to generate site-specific antibody conjugates. For example, antibodies containing a recombinant Fc polypeptide provided herein can be produced by cells cultured with a carbohydrate containing a first functional group (e.g., an azide-functionalized ManNAc analog) such that the first functional group is conjugated to the recombinant Fc polypeptide (e.g., to generate functionalized antibodies), and the functionalized antibodies can be contacted with one or more molecules each including a second functional group that is reactive with the first functional group (e.g., an azide-reactive functional group) such that the first functional group and the second functional group can ligate the functionalized antibodies with the molecule(s) to form site-specific antibody conjugates.

Having the ability to producing polypeptide conjugates (e.g., antibody conjugates) as described herein (e.g., by coupling protein engineering with metabolic glycoengineering such as by using a recombinant polypeptide provided herein (e.g., a recombinant Fc polypeptide that comprises, consists essentially of, or consists of an amino acid sequence set forth in any one of SEQ ID NOs:3-8) provides a simple, versatile, and site-specific workflow to produce chemistry-functionalized antibodies without the need for extensive or harsh modifications. The methods and materials provided herein enable rapid production of antibody-conjugates as well as the formation of more homogenous and chemically defined products for biomedical and industrial applications.

In general, one aspect of this document features recombinant Fc polypeptides that include an engineered N-linked glycosylation site. The recombinant The Fc polypeptide can be generated from a mammalian Fc polypeptide. The recombinant Fc polypeptide can be generated from a human Fc polypeptide. The recombinant Fc polypeptide can comprise or consist essentially of an amino acid sequence set forth in any one of SEQ ID NOs:3-8.

In another aspect, this document features nucleic acid constructs that can encode a recombinant Fc polypeptide, where the recombinant Fc polypeptide includes an engineered N-linked glycosylation site. The Fc polypeptide can be generated from a mammalian Fc polypeptide. The Fc polypeptide can be generated from a human Fc polypeptide. The recombinant Fc polypeptide can comprise or consist essentially of an amino acid sequence set forth in any one of SEQ ID NOs:3-8. The construct can be in the form of a vector.

In another aspect, this document features antibodies including a recombinant Fc polypeptide, where the recombinant Fc polypeptide includes an engineered N-linked glycosylation site. The recombinant Fc polypeptide can be generated from a mammalian Fc polypeptide. The recombinant Fc polypeptide can be generated from a human Fc polypeptide. The recombinant Fc polypeptide can comprise or consist essentially of an amino acid sequence set forth in any one of SEQ ID NOs:3-8. The antibody can target an antigen present on a mammalian cell. The antibody can target an antigen present on a human cell.

In another aspect, this document features methods for making an antibody conjugate. The methods can include, or consist essentially of, (a) contacting an antibody comprising the recombinant Fc polypeptide, where the recombinant Fc polypeptide includes an engineered N-linked glycosylation site with a composition including a carbohydrate comprising a first functional group to incorporate the first functional group into the engineered N-linked glycosylation site of the recombinant Fc polypeptide thereby generating a functionalized antibody; and (b) contacting the functionalized antibody with a molecule comprising a second functional group to conjugate the molecule to the functionalized antibody; thereby generating the antibody conjugate. The Fc polypeptide can be generated from a mammalian Fc polypeptide. The Fc polypeptide can be generated from a human Fc polypeptide. The recombinant Fc polypeptide can comprise or consist essentially of an amino acid sequence set forth in any one of SEQ ID NOs:3-8. The carbohydrate can be a monosaccharide analog. The carbohydrate can be N-acetylmannosamine (ManNAc), N-acetylgalactosamine (GalNAc), N-acetylglucosamine (GlcNAc), fucose, or any analog thereof. The first functional group can be an azide group and the second functional group can be an azide-reactive functional group. The carbohydrate including the first functional group can be a 1,3,4-O-Bu₃ManNAz. The azide-reactive functional group can be an alkyne group, a dibenzocyclooctyne (DBCO) group, a bicyclononyne (BCN) group, or a triphenylphosphine group. The first functional group can be a thiol group the second functional group is a thiol-reactive functional group. The thiol-reactive functional group can be a maleimide group, acrylamides, vinyl sulfone, acetyl bromide, acetyl iodine, pyridyl disulfide, or tosylated linkers. The first functional group can be a ketone group and the second functional group can be a ketone-reactive functional group. The ketone-reactive functional group can be a hydrazide group or an aminooxy groups. The first functional group can be an aldehyde group and the second functional group can be an aldehyde-reactive functional group. The aldehyde-reactive functional group can be a hydrazide group or an aminooxy group. The molecule can be a small molecule, a nucleic acid, a polypeptide, or a peptide-oligonucleotide conjugate (POC). The molecule can be an imaging agent or a therapeutic agent. The antibody including the recombinant Fc polypeptide can be produced by a cell in a cell culture, where the cell includes a nucleic acid construct encoding the antibody such that the cell expresses the antibody, and where contacting the antibody with the composition includes supplementing culture media of the cell culture with the composition. The carbohydrate including the functional group can be present in the culture media at a concentration of from about 1 μM to 1 mM.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1D: Schematics of Fc glycovariants and the manufacturing process. FIG. 1A) Crystallographic structure of human IgG1 Fc (PDB ID: 5jii), with the engineered glycovariant sites and the canonical N297 glycan site indicated. FIG. 1B) Fc glycovariants were manufactured by supplementing the culture media with an azide-functionalized ManNAc analog. Nonreducing (FIG. 1C) and reducing (FIG. 1D) SDS-PAGE analyses of WT, N297, and engineered Fc glycovariants are shown.

FIGS. 2A-2F: Fc glycovariants expressed robustly and incorporated azides to allow fluorescent labeling, while retaining antigen and Fc receptor binding. FIG. 2A) Bar plot showing yield of antibodies from human embryonic kidney (HEK) 293F cells following purification with protein G. FIG. 2B) Bar plot showing average number of dye molecules per antibody molecule, determined by dye/protein ratio of azide-functionalized wild type (WT) F5111 antibody and glycovariants thereof labeled with dibenzocyclooctyne (DBCO)-linked fluorescent dye, as measured by UV/Vis spectroscopy. FIG. 2C) Biolayer interferometry studies of the equilibrium binding between immobilized IL-2 and soluble F5111 glycovariants. FIG. 2D) Biolayer interferometry studies of the equilibrium binding between immobilized FcRn and soluble F5111 glycovariants. Biolayer interferometry studies of the equilibrium binding between immobilized FcγRI (FIG. 2E) or FcγRIIa (FIG. 2F) and soluble F5111 glycovariants with re-introduced N297 glycosylation site.

FIGS. 3A-3B: Fc glycosylation sites were grafted onto antibodies of distinct specificity for fluorescent labeling. FIG. 3A) Average number of dye molecules per antibody molecule, determined by dye/protein ratio of azide-functionalized wild type (WT) 1d3 antibody and glycovariants thereof labeled with DBCO-linked fluorescent dyes, as measured by UV/Vis spectroscopy. FIG. 3B) Mouse CD19⁺ A20 B cell staining of fluorescently labeled 1d3 glycovariant antibodies, as detected via flow cytometry. CD19− (HEK 293F) cells were used as a negative control.

FIGS. 4A-4C: Azide incorporation in Fc glycovariants was optimized through use of different analogs, production in alternative cell lines, and incorporation of multiple glycosylation sites. FIG. 4A) Average number of dye molecules per antibody molecule, determined by dye/protein ratio of azide-functionalized and DBCO-linked fluorescent dye-labeled S4 F5111 glycovariant antibody produced with varying amounts of 1,3,4-O-Bu3ManNAz (ManNAz) or Bu4GalNAz (GalNAz) in HEK 293F or Chinese hamster ovary (CHO)-S cells, as measured by UV/Vis spectroscopy. FIG. 4B) Reducing SDS-PAGE analysis of wild type (WT) F5111 antibody and glycovariants thereof, including double and triple glycomutant antibodies. FIG. 4C) Average number of dye molecules per antibody molecule, determined by dye/protein ratio of azide-functionalized WT F5111 antibody and double or triple mutant glycovariants thereof labeled with DBCO-linked fluorescent dye, as measured by UV/Vis spectroscopy. HC, heavy chain; LC, light chain.

FIGS. 5A-5B: Schematics showing exemplary Fc glycoengineering strategies. FIG. 5A) Illustration of current site-specific conjugation approaches using metabolic incorporation of commercial ManNAz analogs into the canonical Fc glycan. FIG. 5B) Illustration of using metabolic incorporation of butyrated ManNAz analogs into engineered Fc glycan sites as described herein.

FIGS. 6A-6E: Fc glycovariants demonstrated potent tumor cell killing upon formulation as antibody-drug conjugates. FIG. 6A) Biolayer interferometry studies of the equilibrium binding between immobilized human epidermal growth factor receptor 2 (HER2) and soluble trastuzumab glycovariant antibodies. FIG. 6B) Cytotoxicity of single mutant trastuzumab glycovariant antibody-drug conjugates (ADCs) against HER2-expressing SKBR3 human breast cancer cells. DBCO-linked monomethyl auristatin E (MMAE) was used as the drug payload only control. Cytotoxicity of a single and triple mutant trastuzumab glycovariant ADCs against HER2+ SKBR3 (FIG. 6C), HER2+ HCC1954 (FIG. 6D), or HER2− MDA-MB-231 (FIG. 6E) human breast cancer cells. DBCO-linked MMAE was used as the drug payload only control. Error bars represent standard deviation (n=4).

FIGS. 7A-7F: Fc glycovariant conjugation to biomaterials enabled targeted gene delivery. FIG. 7A) Schematic of detection scheme for flow cytometry analysis of azido-modified trastuzumab glycovariants linked to DBCO-coated magnetic microparticles. Antibody conjugation was detected with a fluorescent anti-Fab antibody and target antigen binding was detected using biotinylated HER2 and secondary fluorescent streptavidin staining. Representative flow cytometry plots are shown below. FIG. 7B) Cartoon depicting poly(beta-amino ester) (PBAE) nanoparticle encapsulating cyanine-5 (Cy5)-labeled enhanced green fluorescent protein (eGFP)-encoding mRNA conjugated to trastuzumab glycovariant antibodies. Nanoparticle uptake in transfected HER2+ SKBR3 (FIG. 7C) and HER2− MDA-MB-231 (FIG. 7D) cells, normalized to the unconjugated control for each concentration. eGFP expression in transfected SKBR3 (FIG. 7E) and MDA-MB-231 (FIG. 7F) cells, normalized to the unconjugated control for each concentration. Statistical significance was determined by two-way ANOVA with a Dunnett post hoc test. *p≤0.05, **p≤0.01, ***p≤0.001, and ****p≤0.0001 (n=4).

FIG. 8: Sequence layout of engineered Fc glycovariants (SEQ ID NO:9). Annotated amino acid sequence of heavy chain constant 2 (CH2) and CH3 regions of human immunoglobulin G1 (IgG1) fragment crystallizable (Fc) domain, with canonical N297 glycan and engineered glycovariant sites indicated.

FIG. 9: Production of 1d3 antibody and derivative Fc glycovariants. Nonreducing and reducing SDS-PAGE analysis of the 1d3 antibody in wild type (WT) and engineered Fc glycovariant formats. HC, heavy chain; LC, light chain.

FIGS. 10A-10C: Trastuzumab glycovariants were produced with high purity and incorporated azides to allow fluorescent labeling. FIG. 10A) Nonreducing and reducing SDS-PAGE analysis of the trastuzumab antibody in N297G, and engineered Fc glycovariant formats. FIG. 10B) Analytical high-performance liquid chromatography (HPLC) traces of the trastuzumab antibody glycovariants. FIG. 10C) Average number of dye molecules per antibody molecule, determined by dye/protein ratio of azide-functionalized trastuzumab glycovariant antibodies labeled with dibenzocyclooctyne (DBCO)-linked fluorescent dyes, as measured by UV/Vis spectroscopy. HC, heavy chain; LC, light chain.

FIGS. 11A-11C: Fc glycovariants demonstrated potent and specific tumor cell killing upon formulation as antibody-drug conjugates. Cytotoxicity of single site trastuzumab glycovariant ADCs against HER2+ MDA-MB-453 (FIG. 11A), HER2+ HCC1954 (FIG. 11B), or HER2− MDA-MB-231 (FIG. 11C) human breast cancer cells. DBCO-linked monomethyl auristatin E (MMAE) was used as the drug payload only control. Error bars represent standard deviation (n=4).

FIGS. 12A-12C: Fc glycovariants showed promising developability properties. FIG. 12A) Plasma stability studies of azide-functionalized trastuzumab glycovariant antibodies labeled with DBCO-linked fluorescent dye. Error bars represent standard deviation (n=2). FIG. 12B) Thermal shift assays for determination of melting temperatures of trastuzumab glycovariant antibodies in unconjugated and MMAE-conjugated ADC formats. FIG. 12C) Dynamic-light-scattering (DLS) analysis of trastuzumab glycovariant antibodies.

FIGS. 13A-13C: Fc glycoconjugates were formed via sialic acid linkages and were resistant to human sialidase. FIG. 13A) In-gel fluorescence analysis of DBCO-linked fluorescent dye-labeled trastuzumab glycovariant antibodies following treatment with bacterial sialidase and PNGase. FIG. 13B) In-gel fluorescence of azido-modified trastuzumab glycovariant antibodies pre-treated with bacterial sialidase then subsequently conjugated to DBCO-linked fluorescent dye. FIG. 13C) In-gel fluorescence of DBCO-linked fluorescent dye-labeled trastuzumab glycovariant antibodies following treatment with human neuraminidase 1.

### DETAILED DESCRIPTION

This document provides methods and materials for producing polypeptide conjugates (e.g., antibody conjugates). In some cases, this document provides recombinant polypeptides (e.g., recombinant Fc polypeptides) that include one or more (e.g., one, two, three, four, or more) N-linked glycosylation sites. In some cases, recombinant polypeptides provided herein (e.g., recombinant Fc polypeptides) can be functionalized. For example, polypeptides including one or more recombinant polypeptides provided herein (e.g., antibodies including a recombinant Fc polypeptide provided herein) can be contacted (e.g., in cell culture during cellular translation) with one or more carbohydrates each containing a first functional group to produce functionalized recombinant polypeptides (e.g., functionalized antibodies). In some cases, recombinant polypeptides provided herein (e.g., recombinant Fc polypeptides that comprise, consist essentially of, or consist of an amino acid sequence set forth in any one of SEQ ID NOs:3-8) can be used to generate polypeptide conjugates. For example, polypeptides including one or more recombinant polypeptides provided herein (e.g., antibodies including a recombinant Fc polypeptide that comprises, consists essentially of, or consists of an amino acid sequence set forth in any one of SEQ ID NOs:3-8) can be contacted (e.g., in cell culture during cellular translation) with one or more carbohydrates each containing a first functional group to produce functionalized recombinant polypeptides (e.g., functionalized antibodies) and the functionalized recombinant polypeptides (e.g., the functionalized antibodies) can be contacted with one or more molecules each including a second functional group that is reactive with the first functional group such that the first functional group and the second functional group can ligate the functionalized recombinant polypeptides with the molecule(s) to form polypeptide conjugates (e.g., antibody conjugates).

In some cases, the methods and materials provided herein do not include any copper.

A recombinant polypeptide provided herein (e.g., a recombinant Fc polypeptide that comprises, consists essentially of, or consists of an amino acid sequence set forth in any one of SEQ ID NOs:3-8) can be derived from any appropriate Fc polypeptide. A Fc polypeptide that can be used to generate a recombinant polypeptide provided herein can be a mammalian Fc polypeptide. For example, a Fc polypeptide can be a human Fc polypeptide. Examples of Fc polypeptides include, without limitation, Fc polypeptides derived from an IgG second heavy chain constant (CH2) domain, Fc polypeptides derived from an IgG third heavy chain constant (CH3) domain, Fc polypeptides derived from an IgA CH2 domain, Fc polypeptides derived from an IgA CH3 domain, Fc polypeptides derived from an IgD CH2 domain, Fc polypeptides derived from an IgD CH3 domain, Fc polypeptides derived from an IgM CH2 domain, Fc polypeptides derived from an IgM CH3 domain, Fc polypeptides derived from an IgM fourth heavy chain constant (CH4) domain, Fc polypeptides derived from an IgE CH2 domain, Fc polypeptides derived from an IgE CH3 domain, and Fc polypeptides derived from an IgE CH4 domain.

In some cases, a Fc polypeptide that can be used to generate a recombinant polypeptide provided herein (e.g., a recombinant Fc polypeptide that comprises, consists essentially of, or consists of an amino acid sequence set forth in any one of SEQ ID NOs:3-8) can have an amino acid set forth in The UniProt Knowledgebase (UniProtKB; see, e.g., The UniProt Consortium, Nucleic Acids Research, 51 (D1): D523-D531 (2023)) at, for example, P01857 IGHG1_HUMAN, P01859 IGHG2_HUMAN, P01860 IGHG3_HUMAN, P01861 IGHG4_HUMAN, P01854 IGHE_HUMAN, P01880 IGHD_HUMAN, P01871 IGHM_HUMAN, P01876 IGHA1_HUMAN, and P01877 IGHA2_HUMAN. For example, a Fc polypeptide that can be used to generate a recombinant polypeptide provided herein can have the amino acid sequence set forth in Table 1.

TABLE 1
Exemplary Fc polypeptides that can be used to generate a recombinant polypeptide
provided herein. The canonical N297 sequon (the sequon including an “N” at residue 297
according to the “EU Numbering” scheme) is

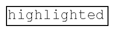

| | Polypeptide Sequence | SEQ ID NO: |
|---|---|---|
| hIgG1 WT | APELLGGPSVFLFPPKPKDTLMISRTPEVTCVVVDVSHEDPEVKFN | 1 |
| | 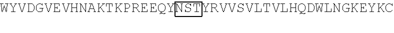 | |
| | KVSNKALPAPIEKTISKAKGQPREPQVYTLPPSREEMTKNQVSLTC | |
| | LVKGFYPSDIAVEWESNGQPENNYKTTPPVLDSDGSFFLYSKLTVD | |
| | KSRWQQGNVFSCSVMHEALHNHYTQKSLSLSPGK | |
| hIgG1 N297X | APELLGGPSVFLFPPKPKDTLMISRTPEVTCVVVDVSHEDPEVKFN | 2 |
| | 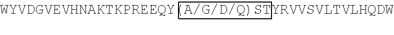 | |
| | LNGKEYKCKVSNKALPAPIEKTISKAKGQPREPQVYTLPPSREEMT | |
| | KNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |

In some cases, a Fc polypeptide that can be used to generate a recombinant polypeptide provided herein (e.g., a recombinant Fc polypeptide that comprises, consists essentially of, or consists of an amino acid sequence set forth in any one of SEQ ID NOs:3-8) can have a glycine (G) at residue 297 (e.g., such that the 297 residue cannot be glycosylated).

In some cases, a Fc polypeptide that can be used to generate a recombinant polypeptide provided herein (e.g., a recombinant Fc polypeptide that comprises, consists essentially of, or consists of an amino acid sequence set forth in any one of SEQ ID NOs:3-8) can have an alanine (A) at residue 297 (e.g., such that the 297 residue cannot be glycosylated).

In some cases, a Fc polypeptide that can be used to generate a recombinant polypeptide provided herein (e.g., a recombinant Fc polypeptide that comprises, consists essentially of, or consists of an amino acid sequence set forth in any one of SEQ ID NOs:3-8) can have an aspartic acid (D) at residue 297 (e.g., such that the 297 residue cannot be glycosylated).

In some cases, a Fc polypeptide that can be used to generate a recombinant polypeptide provided herein (e.g., a recombinant Fc polypeptide that comprises, consists essentially of, or consists of an amino acid sequence set forth in any one of SEQ ID NOs:3-8) can have a glutamine (Q) at residue 297 (e.g., such that the 297 residue cannot be glycosylated).

In some cases, a Fc polypeptide that can be used to generate a recombinant polypeptide provided herein (e.g., a recombinant Fc polypeptide that comprises, consists essentially of, or consists of an amino acid sequence set forth in any one of SEQ ID NOs:3-8) can have an asparagine (N) at residue 297 (e.g., such that the 297 residue can be glycosylated).

A recombinant polypeptide provided herein (e.g., a recombinant Fc polypeptide that comprises, consists essentially of, or consists of an amino acid sequence set forth in any one of SEQ ID NOs:3-8) can be any appropriate length (e.g., can include any number of amino acids). In some cases, a peptide can be from about 200 amino acids in length to about 350 amino acids in length (e.g., from about 200 to about 325, from about 200 to about 300, from about 200 to about 275, from about 200 to about 250, from about 200 to about 225, from about 225 to about 350, from about 250 to about 350, from about 275 to about 350, from about 300 to about 350, from about 325 to about 350, from about 225 to about 325, from about 250 to about 300, from about 225 to about 275, from about 250 to about 300, from about 275 to about 325, about 217, or about 325 amino acids in length).

A recombinant polypeptide provided herein (e.g., a recombinant Fc polypeptide that comprises, consists essentially of, or consists of an amino acid sequence set forth in any one of SEQ ID NOs:3-8) can have any appropriate amino acid sequence. In some cases, a recombinant polypeptide provided herein can have one or more (e.g., one, two, three, four, five, or more) amino acid modifications (e.g., substitutions) relative to a Fc polypeptide set forth in SEQ ID NO:1 or SEQ ID NO:2. In some cases, a recombinant polypeptide provided herein have (e.g., can be engineered to have) one or more sequons (e.g., one or more amino acid sequences that can serve as an attachment site for a glycan such as an N-linked-glycan). A sequon that can be included in a recombinant polypeptide provided herein can be any appropriate sequon. In some cases, a sequon can have the amino acid sequence Asn-X-Ser/Thr, where X can be any amino acid except proline. A sequon that can be included in a recombinant polypeptide provided herein can be at any appropriate location within the recombinant polypeptide. For example, a sequon can be included in a recombinant polypeptide provided herein at a location that is sterically available for click-chemistry conjugation reactions. In cases, where a recombinant polypeptide provided herein is generated from a sequence set forth in SEQ ID NO:1 or SEQ ID NO:2, a sequon included an amino acid residue at any one or more of positions 252-257, 280-294, 307-318, 326-347, 355-362, 384-392, 400-403, 413-421, and/or 432-447, according to the “EU Numbering” scheme.

In some cases, a recombinant polypeptide provided herein (e.g., a recombinant Fc polypeptide) can comprise, consist essentially of, or consist of an amino acid sequence set forth in Table 2.

TABLE 2
Exemplary recombinant Fc polypeptides with the engineered N-glycan sequons

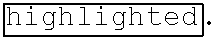

| | Polypeptide Sequence | SEQ ID NO: |
|---|---|---|
| hIgG1 S1 | APELLGGPSVFLFPPKPKDTLMISRTPEVTCVVVDVSHEDPEVKF | 3 |
| | 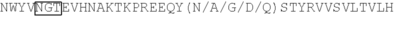 | |
| | QDWLNGKEYKCKVSNKALPAPIEKTISKAKGQPREPQVYTLPPSR | |
| | EEMTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| | GK | |
| hIgG1 S2 | 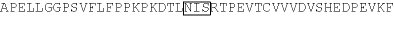 | 4 |
| | NWYVDGVEVHNAKTKPREEQY (N/A/G/D/Q) STYRVVSVLTVLH | |
| | QDWLNGKEYKCKVSNKALPAPIEKTISKAKGQPREPQVYTLPPSR | |
| | EEMTKNQVSLTCLVKGFYPSDIAVEWESNGOPENNYKTTPPVLDS | |
| | DGSFFLYSKLTVDKSRWQQGNVFSCSVMHEALHNHYTQKSLSLSP | |
| | GK | |
| hIgG1 S3 | APELLGGPSVFLFPPKPKDTLMISRTPEVTCVVVDVSHEDPEVKF | 5 |
| | 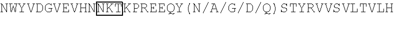 | |
| | QDWLNGKEYKCKVSNKALPAPIEKTISKAKGOPREPQVYTLPPSR | |
| | EEMTKNQVSLTCLVKGFYPSDIAVEWESNGQPENNYKTTPPVLDS | |
| | DGSFFLYSKLTVDKSRWQQGNVFSCSVMHEALHNHYTOKSLSLSP | |
| | GK | |
| hIgG1 S4 | APELLGGPSVFLFPPKPKDTLMISRTPEVTCVVVDVSHEDPEVKF | 6 |
| | NWYVDGVEVHNAKTKPREEQY (N/A/G/D/Q) STYRVVSVLTVLH | |
| | QDWLNGKEYKCKVSNKALPAPIEKTISKAKGQPREPQVYTLPPSR | |
| | EEMTKNQVSLTCLVKGFYPSDIAVEWESNGQPENNYKTTPPVLDS | |
| | 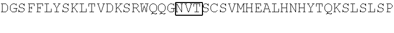 | |
| | GK | |
| hIgG1 S5 | APELLGGPSVFLFPPKPKDTLMISRTPEVTCVVVDVSHEDPEVKF | 7 |
| | NWYVDGVEVHNAKTKPREEQY (N/A/G/D/Q) STYRVVSVLTVLH | |
| | QDWLNGKEYKCKVSNKALPAPIEKTISKAKGQPREPQVYTLPPSR | |
| | EEMTKNQVSLTCLVKGFYPSDIAVEWESNGOPENNYKTTPPVLDS | |
| | 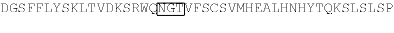 | |
| | GK | |
| hIgG1 S6 | APELLGGPSVFLFPPKPKDTLMISRTPEVTCVVVDVSHEDPEVKF | 8 |
| | NWYVDGVEVHNAKTKPREEQY (N/A/G/D/Q) STYRVVSVLTVLH | |
| | QDWLNGKEYKCKVSNKALPAPIEKTISKAKGQPREPQVYTLPPSR | |
| | 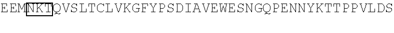 | |
| | DGSFFLYSKLTVDKSRWQQGNVFSCSVMHEALHNHYTQKSLSLSP | |
| | GK | |

A recombinant polypeptide provided herein (e.g., a recombinant Fc polypeptide that comprises, consists essentially of, or consists of an amino acid sequence set forth in any one of SEQ ID NOs:3-8) that consists essentially of an amino acid sequence set forth in Table 2 can include the amino acid sequence set forth in any one of SEQ ID NOs:3-8 with zero, one, two, three, four, five, six, or more amino acid substitutions within the articulated sequence of the sequence identifier (e.g., any one of SEQ ID NOs:3-8), with zero, one, two, three, four, or five amino acid residues preceding the articulated sequence of the sequence identifier (e.g., any one of SEQ ID NOs:3-8), and/or with zero, one, two, three, four, or five amino acid residues following the articulated sequence of the sequence identifier (e.g., any one of SEQ ID NOs:3-8), provided that the recombinant polypeptide retains at least some Fc polypeptide activity (e.g., increasing the hydrodynamic radius of the protein and/or the ability to bind to an Fc receptor).

In some cases, a recombinant polypeptide provided herein (e.g., a recombinant Fc polypeptide that comprises, consists essentially of, or consists of an amino acid sequence set forth in any one of SEQ ID NOs:3-8) can have (e.g., can be engineered to have) two or more (e.g., two, three, four, or more) N-linked glycosylation sites. When a recombinant polypeptide provided herein has two or more N-linked glycosylation sites, the recombinant polypeptide can include any appropriate combination of N-linked glycosylation sites described herein. In some cases, a recombinant polypeptide provided herein can include N-linked glycosylation sites at residues 280 and 421, according to the “EU Numbering” scheme. In some cases, a recombinant polypeptide provided herein can include N-linked glycosylation sites at residues 287 and 421, according to the “EU Numbering” scheme. In some cases, a recombinant polypeptide provided herein can include N-linked glycosylation sites at residues 421 and 359, according to the “EU Numbering” scheme. In some cases, a recombinant polypeptide provided herein can include N-linked glycosylation sites at residues 280, 421, and 359, according to the “EU Numbering” scheme. In some cases, a recombinant polypeptide provided herein can include N-linked glycosylation sites at residues 287, 421, and 359, according to the “EU Numbering” scheme.

In some cases, a recombinant polypeptide provided herein (e.g., a recombinant Fc polypeptide that comprises, consists essentially of, or consists of an amino acid sequence set forth in any one of SEQ ID NOs:3-8) can part of a larger polypeptide that can target and bind an antigen (e.g., an antibody such as a nanobody or a single chain variable fragment (scFv)). A recombinant polypeptide provided herein can be present in any appropriate antibody. A recombinant polypeptide provided herein can be present in an antibody that can target any appropriate antigen. In some cases, a recombinant polypeptide provided herein can be present in a therapeutic antibody. For example, a recombinant polypeptide provided herein can be present in an antibody that can target (e.g., target and bind) an antigen present in a disease cell (e.g., an antigen present in a cancer cell). For example, a recombinant polypeptide provided herein can be present in an antibody that can target (e.g., target and bind) an antigen present in an immune cell (e.g., an immunosuppressive cell). For example, a recombinant polypeptide provided herein can be present in an antibody that can target (e.g., target and bind) a soluble antigen. Examples of antibodies that can include a recombinant polypeptide provided herein and can be used in the methods and materials provided herein include, without limitation, F5111 antibodies, trastuzumab antibodies, gemtuzumab antibodies, brentuximab antibodies, inotuzumab antibodies, polatuzumab antibodies, enfortumab antibodies, sacituzumab antibodies, belantamab antibodies, moxetumomab antibodies, loncastuximab antibodies, tisotumab antibodies, and mirvetuximab antibodies.

In some cases, a recombinant polypeptide provided herein (e.g., a recombinant Fc polypeptide that comprises, consists essentially of, or consists of an amino acid sequence set forth in any one of SEQ ID NOs:3-8) can be fused to a polypeptide that is not an antibody. Examples of polypeptides that can be fused to a recombinant polypeptide provided herein include, without limitation, receptors, cytokines, and enzymes.

This document also provides nucleic acid molecules that can encode a recombinant polypeptide provided herein (e.g., a recombinant Fc polypeptide that comprises, consists essentially of, or consists of an amino acid sequence set forth in any one of SEQ ID NOs:3-8). For example, this document provides nucleic acid molecules encoding a recombinant Fc polypeptide that comprises, consists essentially of, or consists of an amino acid sequence set forth in SEQ ID NO:3. For example, this document provides nucleic acid molecules encoding a recombinant Fc polypeptide that comprises, consists essentially of, or consists of an amino acid sequence set forth in SEQ ID NO:4. For example, this document provides nucleic acid molecules encoding a recombinant Fc polypeptide that comprises, consists essentially of, or consists of an amino acid sequence set forth in SEQ ID NO:5. For example, this document provides nucleic acid molecules encoding a recombinant Fc polypeptide that comprises, consists essentially of, or consists of an amino acid sequence set forth in SEQ ID NO:6. For example, this document provides nucleic acid molecules encoding a recombinant Fc polypeptide that comprises, consists essentially of, or consists of an amino acid sequence set forth in SEQ ID NO:7. For example, this document provides nucleic acid molecules encoding a recombinant Fc polypeptide that comprises, consists essentially of, or consists of an amino acid sequence set forth in SEQ ID NO:8.

In some cases, a nucleic acid that can encode a recombinant polypeptide provided herein (e.g., a recombinant Fc polypeptide that comprises, consists essentially of, or consists of an amino acid sequence set forth in any one of SEQ ID NOs:3-8) can be in the form of a vector (e.g., a viral vector or a non-viral vector).

When a vector including nucleic acid that can encode a recombinant polypeptide provided herein (e.g., a recombinant Fc polypeptide that comprises, consists essentially of, or consists of an amino acid sequence set forth in any one of SEQ ID NOs:3-8) is a viral vector, any appropriate viral vector can be used. Examples viral vectors include, without limitation, retrovirus vectors, lentivirus vectors, adenovirus vectors, and adeno-associated virus vectors.

When a vector that can encode a recombinant polypeptide provided herein (e.g., a recombinant Fc polypeptide that comprises, consists essentially of, or consists of an amino acid sequence set forth in any one of SEQ ID NOs:3-8) is a non-viral vector, any appropriate non-viral vector can be used. In some cases, a non-viral vector can be an expression plasmid (e.g., a cDNA expression vector).

In addition to nucleic acid that can encode a recombinant polypeptide provided herein (e.g., a recombinant Fc polypeptide that comprises, consists essentially of, or consists of an amino acid sequence set forth in any one of SEQ ID NOs:3-8), a vector (e.g., a viral vector or a non-viral vector) can contain one or more regulatory elements operably linked to the nucleic acid that can encode a recombinant polypeptide provided herein. Such regulatory elements can include promoter sequences, enhancer sequences, response elements, signal peptides, internal ribosome entry sequences, polyadenylation signals, terminators, and inducible elements that modulate expression (e.g., transcription or translation) of a nucleic acid. The choice of regulatory element(s) that can be included in a vector depends on several factors, including, without limitation, inducibility, targeting, and the level of expression desired. For example, a promoter can be included in a vector to facilitate transcription of a nucleic acid encoding a nucleoporin polypeptide. A promoter can be a naturally occurring promoter or a recombinant promoter. A promoter can be ubiquitous or inducible (e.g., in the presence of tetracycline), and can affect the expression of a nucleic acid encoding a polypeptide in a general or tissue-specific manner. Examples of promoters that can be used to drive expression of a recombinant polypeptide provided herein in cells include, without limitation, CMV promoters, CAG promoters, EF1a promoters, SV40 promoters, PGK promoters, UBC promoters, beta-actin promoters, and beta-globin promoters. As used herein, “operably linked” refers to positioning of a regulatory element relative to a nucleic acid encoding a polypeptide in such a way as to permit or facilitate expression of the encoded polypeptide. For example, a vector can contain a promoter and nucleic acid that can encode a recombinant polypeptide provided herein. In this case, the promoter is operably linked to a nucleic acid that can encode a recombinant polypeptide provided herein such that it drives expression of the recombinant polypeptide in cells.

Nucleic acid that can encode a recombinant polypeptide provided herein (e.g., a recombinant Fc polypeptide that comprises, consists essentially of, or consists of an amino acid sequence set forth in any one of SEQ ID NOs:3-8) can be produced by techniques including, without limitation, common molecular cloning, polymerase chain reaction (PCR), chemical nucleic acid synthesis techniques, and combinations of such techniques.

Also provided herein are cells (e.g., host cells) containing nucleic acid that can encode a recombinant polypeptide provided herein (e.g., a recombinant Fc polypeptide that comprises, consists essentially of, or consists of an amino acid sequence set forth in any one of SEQ ID NOs:3-8). For example, a cell can be used to replicate and maintain a vector (e.g., a viral vector or a non-viral vector) including nucleic acid that can encode a recombinant polypeptide provided herein. Examples of cells that can be used to replicate and maintain a vector (e.g., a viral vector or a non-viral vector) including nucleic acid that can encode a recombinant polypeptide provided herein include, without limitation, mammalian cells, bacterial cells, plant cells, yeast cells, and insect cells.

In some cases, recombinant polypeptides provided herein (e.g., recombinant Fc polypeptide that comprise, consist essentially of, or consist of an amino acid sequence set forth in any one of SEQ ID NOs:3-8) can be functionalized. For example, a recombinant polypeptide provided herein can have a functional group conjugated to each N-linked glycosylated site in (e.g., engineered into) the recombinant polypeptide.

A functionalized recombinant polypeptide provided herein can contain any appropriate functional group. In some cases, a functional group can be a bioorthogonal functional group. Examples of functional groups that can be present on a recombinant polypeptide provided herein include, without limitation, azide groups, dibenzocyclooctyne (DBCO) groups, bicyclononyne (BCN) groups, alkyne groups, thiol groups, maleimide groups, ketone groups, hydrazide groups, aminooxy groups, and aldehyde groups.

Any appropriate method can be used to functionalize a recombinant polypeptide provided herein. In some cases, a recombinant polypeptide can be contacted with one or more carbohydrates each containing a functional group (e.g., an azide-functionalized ManNAc) in the presence of a glycosyltransferase such that the functionalized carbohydrates are attached to the N-linked glycosylated sites or are attached to an existing N-glycan (e.g., to produce functionalized recombinant polypeptides).

A carbohydrate containing a functional group that can be used in the methods and materials provided herein can contain any appropriate carbohydrate. In some cases, a carbohydrate can be a naturally occurring carbohydrate. In some cases, a carbohydrate can be a synthetic carbohydrate. In some cases, a carbohydrate can be a carbohydrate analog. Examples of carbohydrates that can be used in a in carbohydrate containing a functional group that can be used in the methods and materials provided herein include, without limitation, N-acetylmannosamine (ManNAc), N-acetylgalactosamine (GalNAc), N-acetylglucosamine (GlcNAc), fucose, and any analogs thereof.

In some cases, a functionalized carbohydrate can be modified by one or more short-chained fatty acids (SCFAs). Examples of SFCAs that can be used to modify a functionalized carbohydrate (e.g., a functionalized carbohydrate that can be used to functionalize a recombinant polypeptide provided herein such as a recombinant Fc polypeptide that comprises, consists essentially of, or consists of an amino acid sequence set forth in any one of SEQ ID NOs:3-8) include, without limitation, acetate, propionate, and butyrate.

A carbohydrate containing a functional group can also be referred to as a functionalized carbohydrate. For example, a carbohydrate containing an azide functional group can also be referred to as an azide-functionalized carbohydrate.

Examples of carbohydrates containing a functional group that can be used in the methods and materials provided herein include, without limitation, azide-functionalized carbohydrates (e.g., azide-functionalized ManNAc analogs such as 1,3,4-O-Bu₃ManNAz), alkyne-functionalized carbohydrates (e.g. alkyne-functionalized ManNAc analogs such as 1,3,4-O-Bu₃ManNAl), thiol-functionalized carbohydrates (e,g, thiol-functionalized ManNAc analogs such as Ac₅ManNTProp), and ketone functionalized carbohydrates (e.g. ketone-functionalized ManNAc analogs such as Ac₄ManNLev).

Any appropriate method can be used to contact a recombinant polypeptide provided herein (e.g., a recombinant Fc polypeptide that comprises, consists essentially of, or consists of an amino acid sequence set forth in any one of SEQ ID NOs:3-8) with one or more carbohydrates each containing a functional group (e.g., an azide-functionalized ManNAc analog). For example, a recombinant polypeptide provided herein can be contacted with one or more carbohydrates each containing a functional group in the presence of a glycosyltransferase such that the functionalized carbohydrates, or metabolites thereof, are attached to the N-linked glycosylated sites (e.g., to produce functionalized recombinant polypeptides). In some cases, a recombinant polypeptide provided herein can be functionalized during cellular translation such that the cellular machinery both translates the recombinant polypeptide and attaches the functional group. For example, cells (e.g., host cells) containing nucleic acid that can encode a recombinant polypeptide provided herein can be cultured under conditions where the cells produce the recombinant polypeptide and the cell culture media can contain (e.g., can be supplemented with) one or more functionalized carbohydrates (e.g., 1,3,4-O-Bu₃ManNAz) such that one or more glycosyltransferases can facilitate co-translational or post-translational attachment of the functionalized carbohydrates, or metabolites thereof (e.g. the azido-sialic acid metabolite of 1,3,4-O-Bu₃ManNAz), to the recombinant polypeptides. In some cases, the one or more glycosyltransferases that can facilitate the co-translational attachment of the functionalized carbohydrates to the recombinant polypeptides provided herein can be endogenous to host cells containing nucleic acid that can encode a recombinant polypeptide provided herein. In some cases, the one or more glycosyltransferases that can facilitate the co-translational attachment of the functionalized carbohydrates to the recombinant polypeptides provided herein can be supplemented in the culture media of host cells containing nucleic acid that can encode a recombinant polypeptide provided herein.

A recombinant polypeptide provided herein (e.g., a recombinant Fc polypeptide that comprises, consists essentially of, or consists of an amino acid sequence set forth in any one of SEQ ID NOs:3-8) can be contacted with any appropriate amount of one or more functionalized carbohydrates (e.g., 1,3,4-O-Bu₃ManNAz). For example, a recombinant polypeptide provided herein can be contacted with from about 1 μM to about 1 mM of one or more functionalized carbohydrates. In some cases, from about 1 μM to 1 mM of one or more functionalized carbohydrates can be present in the culture media of host cells containing nucleic acid that can encode a recombinant polypeptide provided herein.

In some cases, a recombinant polypeptide provided herein (e.g., a recombinant Fc polypeptide that comprises, consists essentially of, or consists of an amino acid sequence set forth in any one of SEQ ID NOs:3-8) can be contacted with one or more functionalized carbohydrates (e.g., 1,3,4-O-Bu₃ManNAz) in bolus doses. For example, a recombinant polypeptide provided herein can be contacted with one or more functionalized carbohydrates about every 48 hours.

In some cases, a recombinant polypeptide provided herein (e.g., a recombinant Fc polypeptide that comprises, consists essentially of, or consists of an amino acid sequence set forth in any one of SEQ ID NOs:3-8) can be contacted with one or more functionalized carbohydrates (e.g., 1,3,4-O-Bu₃ManNAz) continuously. For example, one or more functionalized carbohydrates can be continuously infused into cell culture media supporting cells (e.g., host cells) containing nucleic acid that can encode a recombinant polypeptide provided herein.

When a host cell is used to produce a functionalized recombinant polypeptide provided herein (e.g., a functionalized recombinant Fc polypeptide that comprises, consists essentially of, or consists of an amino acid sequence set forth in any one of SEQ ID NOs:3-8), any appropriate type of cell can be used. In some cases, a cell that can be used as a host cell to functionalize a recombinant polypeptide provided herein can contain one or more endogenous glycosyltransferases. In some cases, a cell that can be used as a host cell to functionalize a recombinant polypeptide provided herein can exhibit little to no endogenous sialyltransferase activity. Examples of cells that can be used as host cells to functionalize a recombinant polypeptide provided herein include, without limitation, mammalian cells (e.g., Chinese hamster ovary (CHO) cells (e.g., CHO-S cells), human embryonic kidney (HEK) cells, and NS0 murine myeloma cells), plant cells, yeast cells, and insect cells.

This document also provides methods for using the recombinant polypeptides provided herein (e.g., functionalized recombinant polypeptides that comprise, consist essentially of, or consist of an amino acid sequence set forth in any one of SEQ ID NOs:3-8) to generate polypeptide conjugates. For example, polypeptides including one or more recombinant polypeptides provided herein (e.g., antibodies including a recombinant Fc polypeptide that comprises, consists essentially of, or consists of an amino acid sequence set forth in any one of SEQ ID NOs:3-8) can be contacted (e.g., in cell culture during cellular translation) with one or more carbohydrates each containing a first functional group to produce functionalized recombinant polypeptides (e.g., functionalized antibodies) and the functionalized recombinant polypeptides (e.g., functionalized antibodies) can be contacted with one or more molecules each including a second functional group that is reactive with the first functional group such that the first functional group and the second functional group can ligate the functionalized recombinant polypeptides with the molecule(s) to form polypeptide conjugates (e.g., antibody conjugates). In cases where the functionalized recombinant polypeptides (e.g., functionalized antibodies) including a first functional group are produced by cells in culture, the functionalized recombinant polypeptides can be purified from the culture prior to contacting the functionalized recombinant polypeptides with one or more molecules each including a second functional group that is reactive with the first functional group.

A molecule including a functional group (e.g., a second functional group that is reactive to a first functional group present on a functionalized recombinant polypeptide provided herein such as a recombinant Fc polypeptide that comprises, consists essentially of, or consists of an amino acid sequence set forth in any one of SEQ ID NOs:3-8) can be any appropriate type of molecule. A molecule including a functional group can be a small molecule, a nucleic acid, a polypeptide (e.g., an antibody), or any combination thereof (e.g., a peptide-oligonucleotide conjugate (POC)). In some cases, a molecule including a functional group can be an imaging agent (e.g., a dye). In some cases, a molecule including a functional group can be a therapeutic agent (e.g., an anti-cancer drug such as a cytotoxic agent). Examples of molecules that can include a functional group and can be used in the methods and materials provided herein include, without limitation, MMAE, monomethyl auristatin F (MMAF), mertansine, calicheamicin, SN-38, exatecan, SG3199, Dxd, DM1, DM4, and Pseudomonas exotoxin.

A molecule including a functional group (e.g., a second functional group) can include any appropriate functional group. In some cases, a functional group can be a bioorthogonal functional group. Examples of functional groups that can be used in a carbohydrate containing a functional group that can be used in the methods and materials provided herein include, without limitation, azide groups, DBCO groups, BCN groups, alkyne groups, thiol groups, maleimide groups, ketone groups, hydrazide groups, aminooxy groups, and aldehyde groups.

In some cases, the functional group included in a molecule including a functional group (e.g., a second functional group) can be selected to be reactive with a functional group present on a functionalized recombinant polypeptide provided herein (e.g., functionalized recombinant Fc polypeptide that comprises, consists essentially of, or consists of an amino acid sequence set forth in any one of SEQ ID NOs:3-8). For example, when a functionalized recombinant polypeptide provided herein is an azide-functionalized recombinant polypeptide a molecule including a functional group can include an azide-reactive functional group. Examples of azide-reactive functional groups that can be included in a molecule including a functional group and used in the methods and materials provided herein include, without limitation, alkyne groups, DBCO groups, BCN groups, and triphenylphosphine groups. In another example, when a functionalized recombinant polypeptide provided herein is a thiol-functionalized recombinant polypeptide a molecule including a functional group can include a thiol-reactive functional group. Examples of thiol-reactive functional groups that can be included in a molecule including a functional group and used in the methods and materials provided herein include, without limitation, maleimide groups, acrylamides, vinyl sulfone, haloacetyls (e.g., acetyl bromide and acetyl iodine), pyridyl disulfides, and tosylated linkers. In still another example, when a functionalized recombinant polypeptide provided herein is a ketone-functionalized recombinant polypeptide a molecule including a functional group can include a ketone-reactive functional group. Examples of ketone-reactive functional groups that can be included in a molecule including a functional group and used in the methods and materials provided herein include, without limitation, hydrazide groups and aminooxy groups. In yet another example, when a functionalized recombinant polypeptide provided herein is an aldehyde-functionalized recombinant polypeptide a molecule including a functional group can include an aldehyde-reactive functional group. Examples of aldehyde-reactive functional groups that can be included in a molecule including a functional group and used in the methods and materials provided herein include, without limitation, hydrazide groups and aminooxy groups.

In some cases, a molecule including a functional group (e.g., a second functional group) can be conjugated to or encapsulated within a carrier such as a nanoparticle (e.g., a lipid nanoparticle such as a PEGylated nanoparticle) or microparticle.

In cases where a functionalized recombinant polypeptide provided herein (e.g., a functionalized recombinant Fc polypeptide) includes a thiol group, the thiol-functionalized polypeptide can be contacted with a molecule (e.g., an imaging agent such as a dye and/or a therapeutic agent such as a cytotoxic agent (e.g., MMAE)) including a thiol-reactive functional group (e.g., a maleimide group) such that the thiol group and the thiol-reactive functional group can ligate the thiol-functionalized polypeptide to the molecule to form a polypeptide conjugate including the recombinant Fc polypeptide conjugated to the molecule. For example, an antibody including a recombinant Fc polypeptide provided herein (e.g., a recombinant Fc polypeptide having an amino acid sequence set forth in any one of SEQ ID NOs:3-8) can be contacted (e.g., in cell culture during cellular translation) with one or more carbohydrates each containing a thiol group to produce thiol-functionalized antibodies, and the thiol-functionalized antibodies can be contacted with one or more cytotoxic agents (e.g., MMAE) each including a thiol-reactive functional group (e.g., a maleimide group) such that the thiol group and the thiol-reactive functional group can ligate the thiol-functionalized antibodies with the cytotoxic agent(s) to form antibody conjugates (e.g., antibody-drug conjugates).

The invention will be further described in the following examples, which do not limit the scope of the invention described in the claims.

### EXAMPLES

### Example 1: Production of Site-Specific Antibody Conjugates Using Metabolic Glycoengineering and Engineered Fc Glycovariants

N-linked glycosylation sites can be designed into antibody variable regions (Ludwig et al., MAbs 14:2095704 (2022)). Conjugation to the canonical Fc glycan at N297 of human immunoglobulin G1 (hIgG1) is difficult because this site is “buried” between the two Fc chains in a typical IgG antibody dimer. Previous attempts at using metabolic supplementation of sugar analogs for conjugating to the 297 glycan led to low azide incorporation (Rochefort et al., Glycobiology 24:62-69 (2014)) or still required disulfide reduction of thiolated analogs (Okeley et al., Bioconjugate Chem. 24:1650-1655 (2013)).

This Example describes the design and generation of Fc glycovariants having unique N-linked glycosylation sites. This Example also describes a glycoengineering approach using such Fc glycovariants to generate site-specific antibody conjugates. For example, antibodies that contain an Fc glycovariant can be subjected to a metabolic glycoengineering approach that uses azido-sugar analogs (e.g., to generate azide-functionalized antibodies) and antibody N-glycosylation (e.g., to generate site-specific antibody conjugates). The Fc glycovariants described herein can avoid possible interactions with target antigen binding, especially if large drugs are attached. Moreover, Fc modification is universally applicable, independent of antibody specificity.

### Results

### Selection and Expression of Engineered Glycans

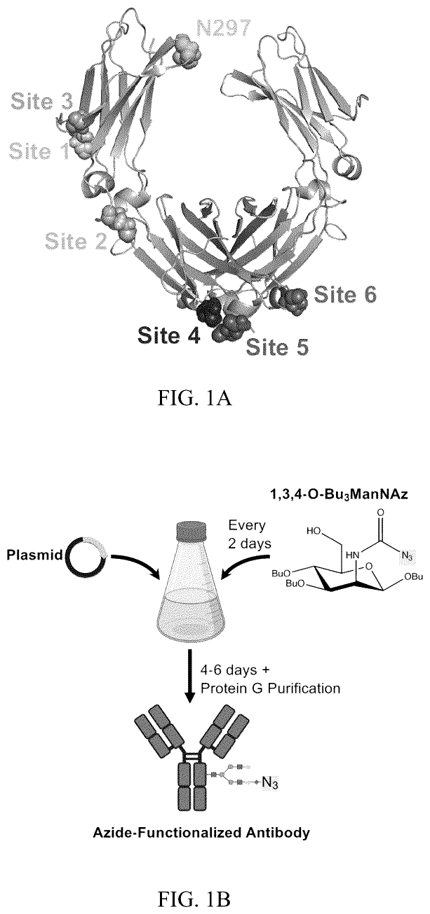

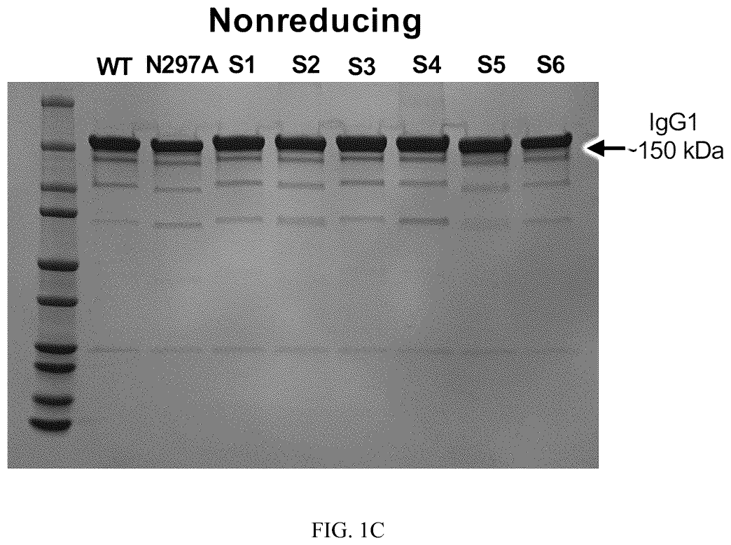

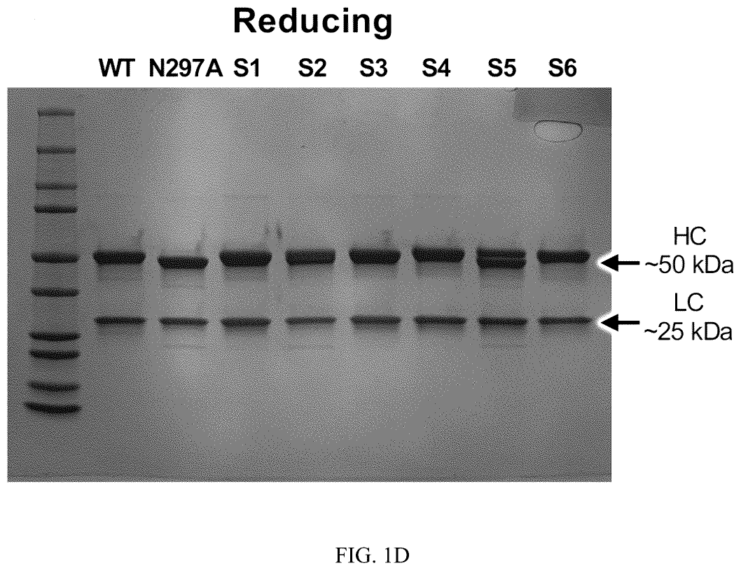

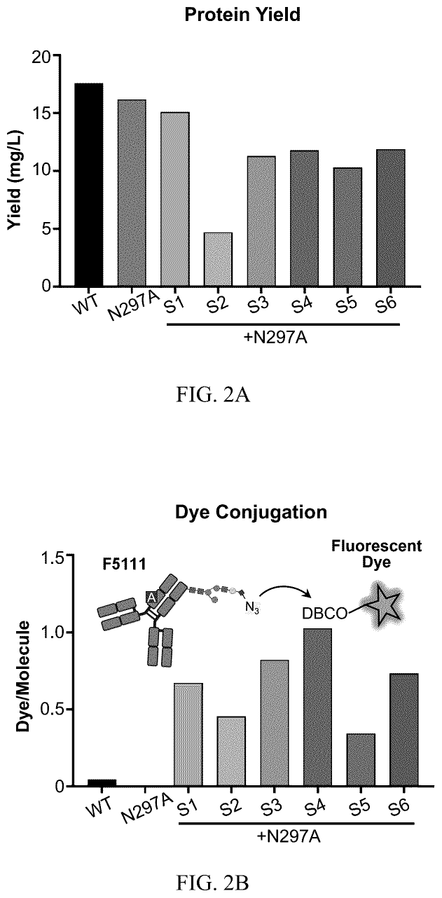

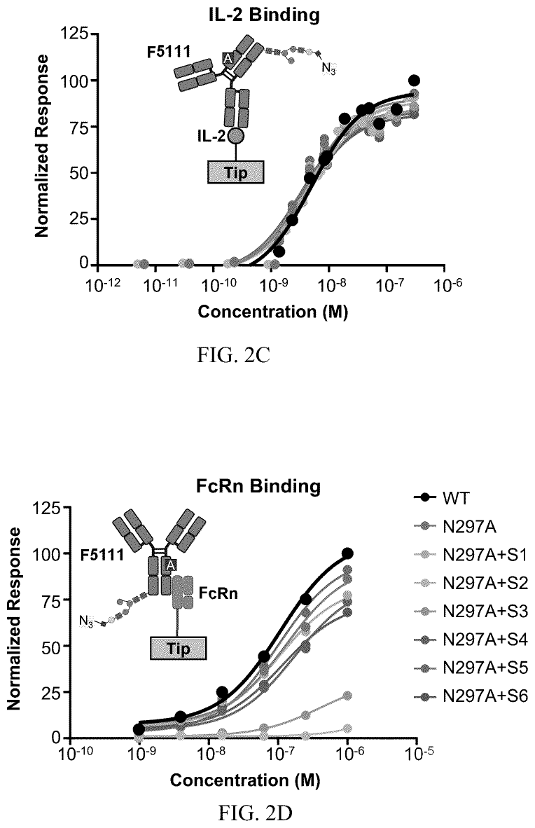

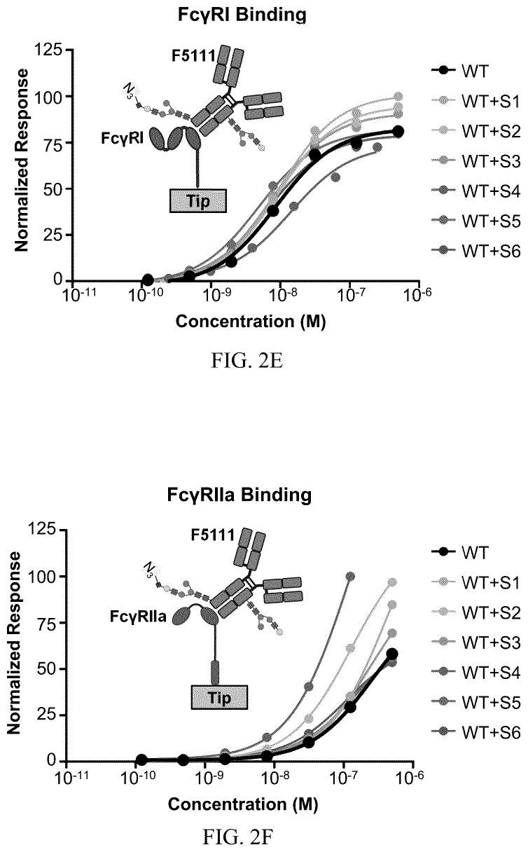

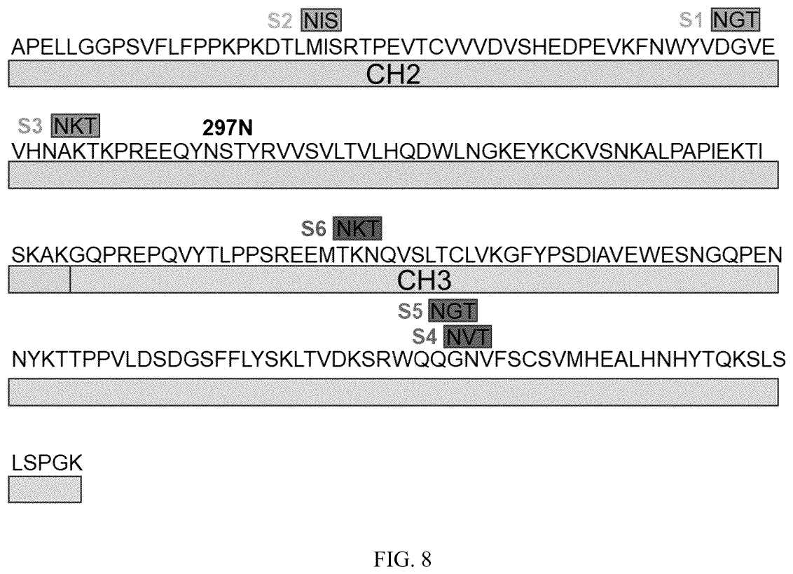

Based on the N-linked glycan design criteria described herein, six sites were chosen to introduce engineered N-glycans (FIGS. 1A and 8, Table 2). The six selected N-glycosylation sites were separately installed into the heavy chain of a representative hIgG1 antibody, the anti-interleukin-2 (IL-2) antibody F5111 (Trotta et al., Nature Medicine 24:1005-1014 (2018)). Each of the resulting F5111 glycovariants also contained the N297A substitution to allow for analysis of the engineered glycosylation sites without confounding effects from the canonical N297 Fc glycan. Each construct was then expressed recombinantly in human embryonic kidney (HEK) 293F cells, with the azido-modified ManNAc analog 1,3,4-O-Bu₃ManNAz (100 mM) supplemented every 2 days (FIG. 1B). The wild type (WT) F5111 antibody, the aglycosylated N297A mutant, and all six glycovariants (denoted S1-S6) expressed as intact IgGs with minimal impurities, as determined by SDS-PAGE analysis (FIG. 1C). Glycosylation of all 6 glycovariants was detected by molecular weight shifts relative to the aglycosylated N297A WT antibody (FIG. 1D). Interestingly, construct S5 showed 2 distinct heavy chain species, likely indicating a mixture of glycosylated and aglycosylated heavy chains. Each construct was expressed recombinantly, and yields were similar to the WT antibody with the exception of S2, which exhibited lower expression (FIG. 2A). Overall, the installed amino acid substitutions successfully introduced glycosylation with minimal effects on antibody purity or yield.

### Engineered Glycovariant Antibodies can be Coupled to Azide Reactive Dye and Retain Functional Properties

The constructs were subjected to dibenzocyclooctyne (DBCO)-dye labeling. The dye/protein ratio was measured as a surrogate for drug-to-antibody-ratio (DAR). All engineered sites exhibited dye incorporation, demonstrating that the engineered glycan sites incorporated azido-sialic acids from 1,3,4-O-Bu3ManNAz supplementation and, furthermore, that these groups were sterically available for click-chemistry conjugation reactions (FIG. 2B). Importantly, all 6 novel sites (which lacked the N297 glycan) exhibited superior labeling compared to the WT antibody (which contained the N297 glycan). Labeling of the N297 glycan was low (FIG. 2B), consistent with previous reports (Okeley et al., Bioconjugate Chem. 24:1650-1655 (2013); and Rochefort et al., Glycobiology 24:62-69 (2014)). This may be due to inaccessibility of the buried site between the two heavy chains in the hIgG1 structure and/or low sialylation. Divergence in labeling between the different F5111 glycovariants could be due to site-to-site variation in glycoforms, which has been observed in the context of various other proteins (Čaval et al., Molecular & Cellular Proteomics 20:100010 (2021)).

To demonstrate that the antibody-antigen binding properties were not disturbed by the Fc mutations used to install the N-glycans, biolayer-interferometry studies were performed against immobilized IL-2, the target protein of the F5111 antibody. The IL-2 binding properties were equivalent to the WT antibody, with a K_{D} value of each glycovariant showing a similar value of ˜4 nM (FIG. 2C, Table 3).

TABLE 3
Equilibrium affinities (Kd) for target antigens.

| Construct | Kd (nM) (95% CI) |
|---|---|
| F5111 WT | 5.1 (3.4, 7.5) |
| F5111 N297A | 4.1 (2.7, 6.4) |
| F5111 N297A + S1 | 4.2 (2.8, 6.6) |
| F5111 N297A + S2 | 5.3 (3.7, 7.5) |
| F5111 N297A + S3 | 4.3 (2.9, 6.6) |
| F5111 N297A + S4 | 4.2 (2.7, 6.4) |
| F5111 N297A + S5 | 3.4 (2.1, 5.5) |
| F5111 N297A + S6 | 4.1 (2.7, 6.3) |
| Trastuzumab N297G | 5.2 (3.5, 7.8) |
| Trastuzumab N297G + S1 | 5.3 (4.5, 6.1) |
| Trastuzumab N297G + S4 | 5.3 (4.5, 6.3) |
| Trastuzumab N297G + S6 | 5.7 (4.7, 6.8) |

An important consideration for antibody pharmacokinetic properties is their ability to bind the neonatal Fc receptor (FcRn) through interactions with the Fc domain, which prolongs half-life by rescuing the antibody from lysosomal degradation following cellular uptake (Roopenian & Akilesh, Nat Rev Immunol 7:715-725 (2007)). Biolayer interferometry-based binding studies against human FcRn demonstrated that 4 of the 6 F5111 glycovariants exhibited similar interaction affinity compared to the WT antibody (FIG. 2D, Table 4). Variants S2 and S3 showed reduced affinity for FcRn, suggesting they could exhibit faster clearance. For cases where Fc effector function is required through interaction with Fcg receptors (FcγR) on immune cells, the glycovariants engineered here would not be appropriate because the N297 glycan is necessary for the Fc domain to bind these receptors (Pincetic et al., Nat Immunol 15:707-716 (2014); and Wright & Morrison, Journal of Experimental Medicine 180:1087-1096 (1994)). To this end, the N297 glycan site was reincorporated into each of the F5111 glycovariants and expressed the corresponding proteins in the presence of 100 μM 1,3,4-O-Bu₃ManNAz. Overall, biolayer interferometry studies revealed that most Fc glycovariants which contained the canonical N297 glycan site showed similar binding responses to FcγRI and FcγRIIa as compared to the WT antibody (FIGS. 2E and F, Table 5). Collectively, these results demonstrate successful incorporation of conjugation-accessible azido-modified sugar analogs into the antibody Fc domain without disruption to the target binding or Fc receptor binding functions of the modified antibodies.

TABLE 4
Equilibrium dissociation constant values
(K_{D}) for antibody/FcRn Binding

| Antibody Construct | K_{D} (nM) (95% CI) for FcRn Binding |
|---|---|
| F5111 WT | 107.4 (50.5, 233) |
| F5111 N297A | 132.5 (41.8, 449.5) |
| F5111 N297A + S1 | 104.7 (40.4, 278.8) |
| F5111 N297A + S2 | ND |
| F5111 N297A + S3 | 398.7 (189.9, 1019) |
| F5111 N297A + S4 | 174.7 (63.7, 521.1) |
| F5111 N297A + S5 | 101.9 (47.5, 222.5) |
| F5111 N297A + S6 | 116.4 (44.7, 312.3) |

TABLE 5
Equilibrium dissociation constant values
(K_{D}) for antibody/FcγRI binding

| Antibody Construct | K_{D} (nM) (95% CI) for FcgRI |
|---|---|
| F5111 WT | 8.5 (5.7, 12.7) |
| F5111 WT + S1 | 9.3 (6.4, 13.6) |
| F5111 WT + S2 | 8.9 (6.4, 12.4) |
| F5111 WT + S3 | 7.5 (5.6, 10.1) |
| F5111 WT + S4 | 14 (6.6, 30.4) |
| F5111 WT + S5 | 4.8 (3.5, 6.8) |
| F5111 WT + S6 | 6.5 (4.8, 8.8) |

### Fc Glycoengineering Approach is Effective Across Antibodies of Distinct Specificities

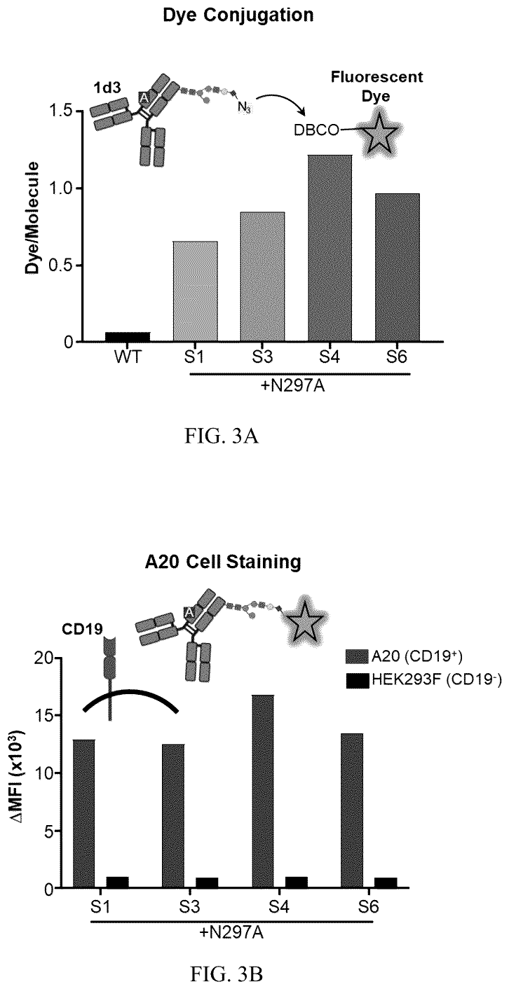

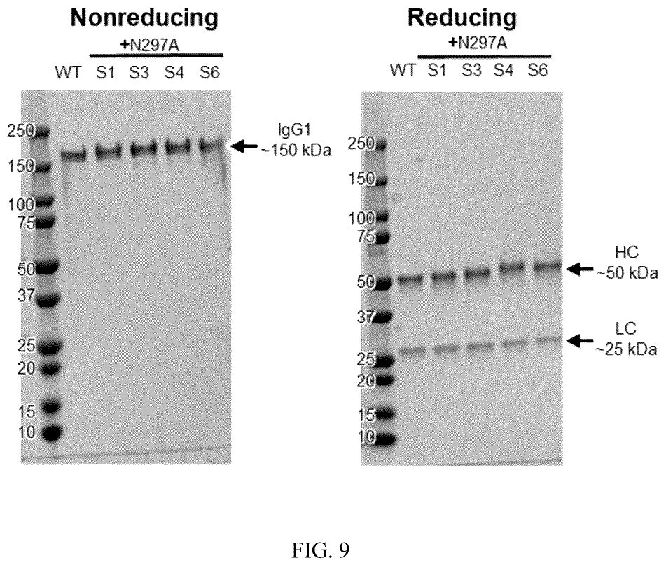

To illustrate the versatility of the Fc glycoengineering approach, it was examined whether the newly installed N-glycan sites could be incorporated into an antibody with a different specificity by grafting the variable domain of the anti-mouse CD19 (mCD19) antibody clone 1d3 (Krop et al., European Journal of Immunology 26:238-242 (1996)) into the hIgG1 backbone. Due to the low yield of the S2 mutant (FIG. 2A) and the incomplete glycosylation of the S5 mutant (FIG. 1D) in the context of the F5111 glycovariants, these mutants were not included in further analyses. The S1, S3, S4, and S6 Fc glycovariant sites were grafted onto a hIgG1 DNA backbone (with the N297A mutation) containing the rat HC variable region of 1d3 and co-transfected these DNA plasmids with a plasmid encoding the rat light chain (LC) variable domain and the human kappa constant domain into HEK 293F cells, supplementing with 100 mM 1,3,4-O-Bu₃ManNAz every 2 days. The harvested 1d3 glycovariants exhibited high purity and showed similar heavy chain molecular weights to the WT 1d3 antibody, which contains the N297 glycan (FIG. 9), suggesting successful glycosylation of the variants. DBCO dye derivative coupling studies showed similar levels of dye conjugation to 1d3 glycovariants compared to those observed for the F5111 glycovariants (FIG. 3A). Furthermore, the 1d3 glycovariants specifically stained the mCD19-expressing A20 mouse B cell line (FIG. 3B), demonstrating that, as in the case of the F5111 antibody, glycoengineering of the 1d3 antibody did not interfere with antigen recognition. Moreover, cell binding further indicated that dye conjugation also did not disrupt antigen binding, highlighting the general applicability of the platform.

### Glycan Incorporation can be Tuned Through Use of Various Analogs, Production in Alternative Cell Lines, and Introduction of Multiple Glycosylation Sites

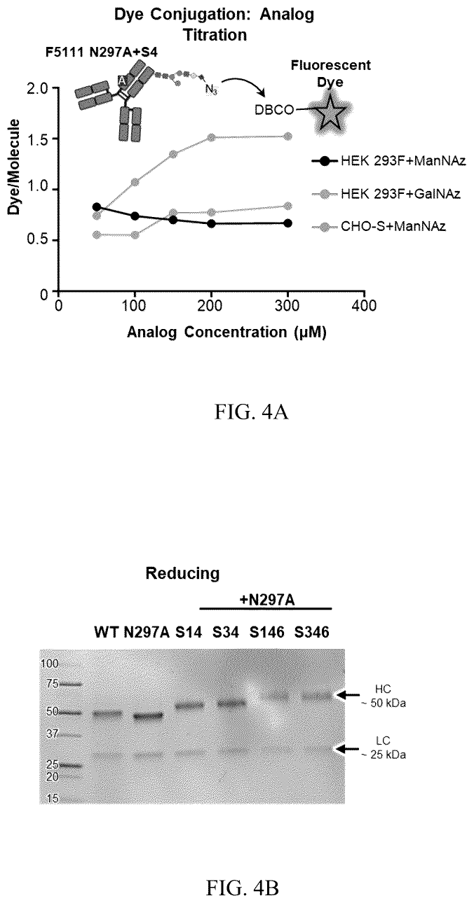

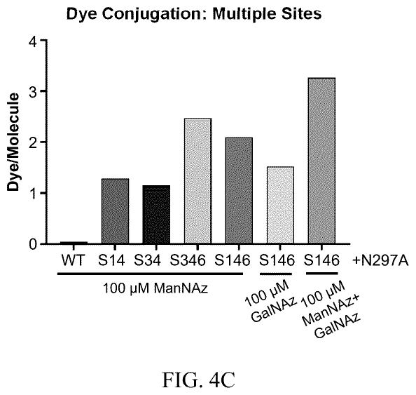

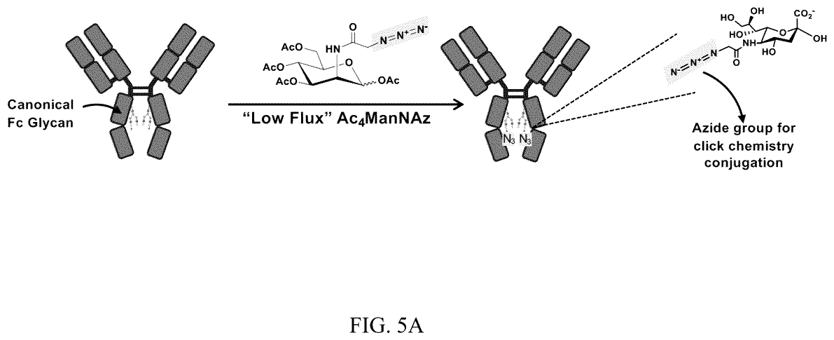

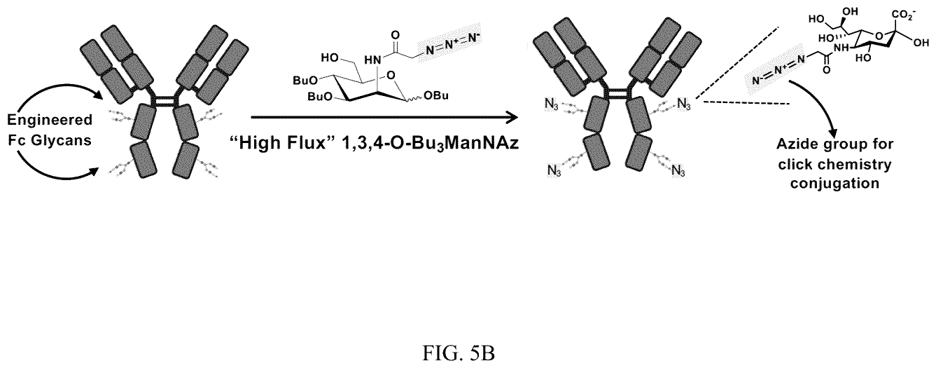

To optimize azido-modified sugar analog incorporation, the concentration of analog during expression of the S4 F5111 glycovariant was titrated. It was noted that in HEK 293F cells, the 100 μM concentration previously implemented for the 1,3,4-O-Bu₃ManNAz analog led to maximal analog incorporation (FIG. 4A), and further improvement was not observed with increasing analog concentrations. Incorporation of a Bu₄GalNAz analog into the S4 F5111 glycovariant was also tested, and it was found that a similar number of azide sites were introduced, reaching saturation at an analog concentration of 150 μM (FIG. 4A). As maximum incorporation for both analogs was below 1 dye molecule per antibody, it was hypothesized that the non-natural sugar analog was likely outcompeted by natural sialic acids being installed into the antibody glycans by the HEK 293F cells. To explore this possibility, the S4 F5111 glycovariant was expressed in the presence of titrated amounts of 1,3,4-O-Bu₃ManNAz in Chinese hamster ovary (CHO)-S cells, which show lower levels of natural sialylation compared to HEK 293F cells (Goh & Ng, Critical Reviews in Biotechnology 38:851-867 (2018)). It was found that glycovariants produced in CHO-S cells reached saturating levels of labeling at an analog concentration of 200 mM. Also, consistent with the hypothesis, it was observed that antibody production in CHO-S cells versus HEK 293F cells leads to incorporation ˜1.5-fold more dye equivalents per protein (FIG. 4A), likely due to the lower endogenous sialylation of CHO-S cells.

To further increase the number of drug conjugation sites, double and triple mutants of the F5111 antibody (containing the N297A substitution) were designed that combined the validated engineered sites for N-glycan installation. Double variants (S14 and S34) and triple variants (S146 and S346) exhibited notably higher molecular weight heavy chains compared to the WT (N297-containing) and N297A mutated F5111 antibodies, consistent with the incorporation of additional glycans (FIG. 4B). Double variants showed a minor increase in extent of conjugation compared to the single variants (>1 dye molecule per antibody), whereas the triple variants demonstrated incorporation of 2 or more dye molecules per antibody (FIG. 4C). To increase incorporation of azides into the engineered glycans, the triple site S146 was expressed in the presence of both the 1,3,4-O-Bu₃ManNAz and Bu₄GalNAz analogs, which led to ˜3 dye molecules per antibody. Overall, these studies demonstrate that the expression system, sugar analogs, and number of engineered sites can all be tuned to manipulate the extent of antibody conjugation.

### Methods

### Novel N-Linked Site Design

Amino acid residues distributed throughout the heavy chain constant domains 2 and 3 (CH2 and CH3) of the human immunoglobulin G1 (hIgG1) fragment crystallizable (Fc) region were chosen for installation of novel sites of N-linked glycosylation. N-linked glycan insertion sites can be created by introduction of a N-X-S/T consensus sequence into a protein, where X is any amino acid except proline (Hart et al., Journal of Biological Chemistry 254:9747-9753 (1979)). A sliding window method was used to identify all potential sites for insertion of this consensus sequence. Theoretical mutations were chosen to either install N-X-T, or N-X-S, whichever required fewer substitutions. Structures of candidate glycovariants were then modelled using PyRosetta, (Chaudhury et al., Bioinformatics 26:689-691 (2010)) based on the wild type hIgG1 Fc structure (PDB ID: 5JII) (Lobner et al., Structure 25:878-889.e5 (2017)). Final sites were chosen according to the following criteria: high solvent exposure, flexible loop secondary structure (determined by DSSP algorithm), minimal disruption to the native sequence, and predicted probability of N-glycosylation >0.6 as determined by the NetNGlyc server (Gupta & Brunak, Pac Symp Biocomput: 310-322 (2002)).

### Analog Design and Antibody Production

Theoretically, various chemical groups can be functionally introduced for conjugation. Azido-modified carbohydrate analogs were selected for use due to the commercial availability of dibenzocyclooctyne (DBCO) moiety-activated molecules, thereby avoiding the need for copper-catalyzed “click chemistry” conjugation reactions. The synthesis of 1,3,4-O-Bu₃ManNAz and has been previously described (Almaraz et al., Biotechnol Bioeng 109:992-1006 (2012); and Saeui et al., Mol Pharm 15:705-720 (2018)). Following synthesis and purification, a concentrated solution (100 mM) of each analog was made using 200 proof ethyl alcohol for long term storage −80° C. On the day of antibody transfection, and every 2 days thereafter, 1,3,4-O-Bu₃ManNAz solution was added to cell cultures at a final concentration of 100 μM unless noted otherwise.

The heavy chain (HC) sequences and light chain (LC) sequences for the hIgG1 lambda F5111 antibody were separately cloned into the gWiz vector (Genlantis) (Trotta et al., Nature Medicine 24:1005-1014 (2018)). Similarly, chimeric HC and LC sequences for an anti-mouse CD19 antibody containing the variable domains of the rat IgG2a 1d3 (Kochenderfer et al., Blood 116:3875-3886 (2010)) antibody and the constant hIgG1 HC and kappa LC domains were separately cloned into the gWiz vector. The HC and LC sequences for the hIgG1 kappa trastuzumab antibody were separately cloned into the gWiz vector (KEGG DRUG: Trastuzumab, (n.d.)). Site-directed mutagenesis to create plasmid vectors containing mutated DNA sequences encoding glycovariant HCs was performed as previously described (H. Liu & Naismith, BMC Biotechnology 8:91 (2008)). For antibody production, the HC and LC DNA plasmids constructs were co-transfected at a 1:1 mass ratio into human embryonic kidney (HEK) 293F cells at a density of 1′10⁶ cells/mL or Expi293F cells at a density of 2′10⁶ cells/mL using polyethylenimine (PEI) MAX (Polysciences). In brief, DNA (1 mg/L culture) and PEI MAX (5.3 mg/L culture) were diluted in Opti-MEM (100 mL/L of culture) (Thermo Fisher), briefly vortexed, and incubated at room temperature for 10 min. Subsequently, the DNA/PEI MAX mixture was added to the culture with gentle swirling. Transfection of Chinese hamster ovary (CHO)-S cells was performed using the poly(beta-amino ester) (PBAE) formulation 4-4-6, as previously described (Luly et al., Int J Nanomedicine 17:4469-4479 (2022)). The day after transfection of Expi293F cells, valproic acid, sodium propionate, and glucose (Sigma) were added to final concentrations of 5 mM, 6.9 mM, and 46 mM, respectively. Secreted antibodies were purified from cell supernatants 4-6 days post-transfection via protein G agarose affinity chromatography.

### Cell Culture

HEK293F, Expi293F, and CHO-S cells (Thermo Fisher) were supplemented with 2 U/mL penicillin, 2 mg/mL streptomycin (Gibco) and cultured in serum-free media according to the manufacturer's instructions. A20 B cells were cultured in ATCC-Modified RPMI-1640 (Thermo Fisher) supplemented with 10% fetal bovine serum (FBS), 100 U/mL penicillin, 100 mg/mL streptomycin (Gibco), and 0.05 mM 2-mercaptoehanol. SKBR3 cells were cultured in McCoy's 5a Medium (Thermo Fisher) supplemented with 10% FBS, and 100 U/mL penicillin, 100 mg/mL streptomycin (Gibco). MDA-MB-453 and MDA-MB-231 cells were cultured in Leibovit's L-15 Medium (Thermo Fisher) supplemented with 10% FBS, and 100 U/mL penicillin, 100 mg/mL streptomycin (Gibco). HCC1954 cells were cultured in ATCC-Modified RPMI-1640 (Thermo Fisher) supplemented with 10% FBS and 100 U/mL penicillin-streptomycin (Gibco). All cell lines were maintained at 37° C. in a humidified atmosphere with 5% CO2.

### Fluorescent Dye and Drug Conjugation

Purified antibodies were conjugated overnight at 4° C. in phosphate-buffered saline (PBS, pH 7.4) containing 20 molar excess of BP Fluor 647 DBCO (BroadPharm). Excess dye was removed through repeated buffer exchange in a 30 kDa MWCO centrifugal filter (Amicon). The Dye/Protein ratio was then quantified using UV/Vis spectroscopy with a NanoDrop (ε_{8648 nm,dye}=270,000 M⁻¹ cm⁻¹). For conjugation of MMAE, DBCO-PEG4-Val-Cit-MMAE (BroadPharm) was similarly incubated with antibodies in 20 molar excess overnight and removed through centrifugal filtration as described above.

### Binding Studies

For biolayer interferometry studies using an Octet® instrument, biotinylated human interleukin-2 (IL-2), produced via HEK 293F cell secretion, as previously described (Ludwig et al., MAbs 14:2095704 (2022.)), human neonatal Fc receptor (FcRn) (Sino Biological), human Fcγ receptor I (FcgRI) (Sino Biological), human FcγRIIa (Sino Biological), and human epidermal growth factor receptor 2 (HER2) (Sino Biological) were immobilized to streptavidin-coated biosensors (Sartorius). For IL-2 and HER-2 binding studies, a background buffer of phosphate-buffered saline pH 7.4 containing 0.1% (w/v) bovine serum albumin (PBSA) was used, and tips were regenerated in 0.1 M glycine pH 2.7. For FcRn binding studies, a background buffer of PBSA pH 5.6 was used, and tips were regenerated in PBSA pH 7.4. For FcγR binding studies, a background buffer of PBSA pH 7.4 was used, and tips were regenerated in 0.1 M glycine pH 3.5.

For mouse CD19⁺ (mCD19⁺) cell staining studies, A20 cells were incubated with 1 μg/mL of fluorescently labeled antibody at 4° C. for 20 min. Cells were then washed twice with PBSA and analyzed on a Cytoflex flow cytometer (Beckman Coulter).

### Example 2: Trastuzumab Fc Glycoconjugates as Exemplary Therapeutics

This Example describes the extension of the glycoengineering approach to the FDA-approved anti-human epidermal growth factor receptor 2 (HER2) antibody trastuzumab (Fu et al., Sig Transduct Target Ther 7:1-25 (2022) K. Liu et al., Molecular Cancer 23:62 (2024); and Paul et al., Nat Rev Cancer 24:399-426 (2024)), The cytotoxic effects of trastuzumab-based antibody-drug glycoconjugates are evaluated against various cell-lines. Antibody-nanoparticle glycoconjugates are then evaluated as a gene delivery method. Various developability properties of the antibody glycoconjugates, including plasma stability, melting temperature, and aggregation properties, are evaluated.

### Results

### Engineered Fc Glycovariant Antibodies Enable Design of Therapeutically Effective Antibody-Drug Conjugates

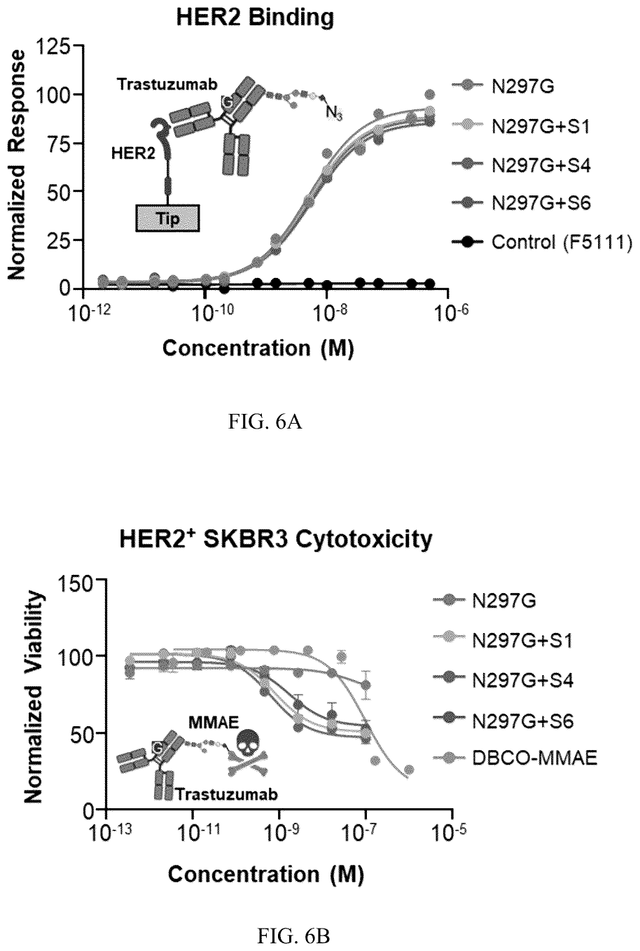

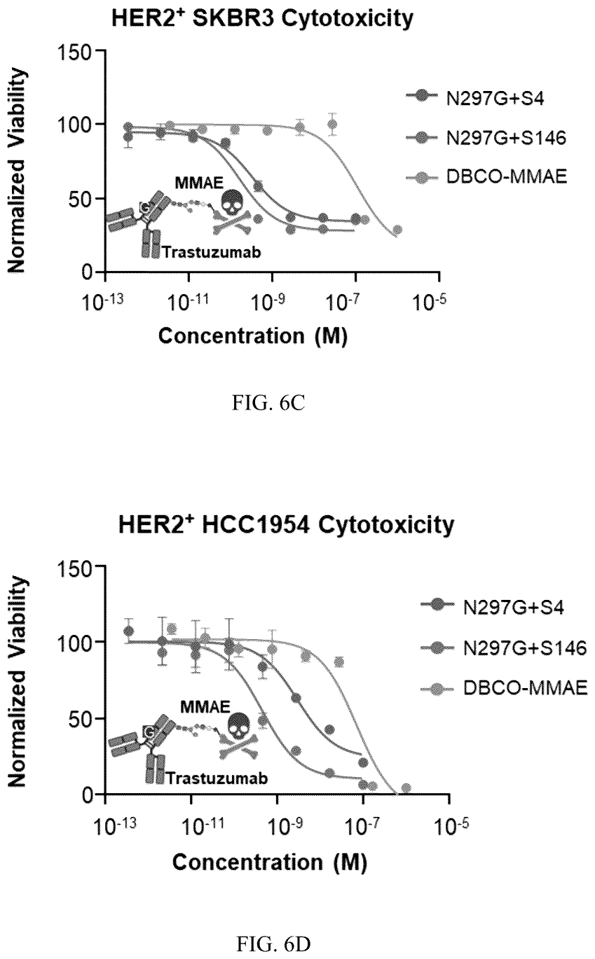

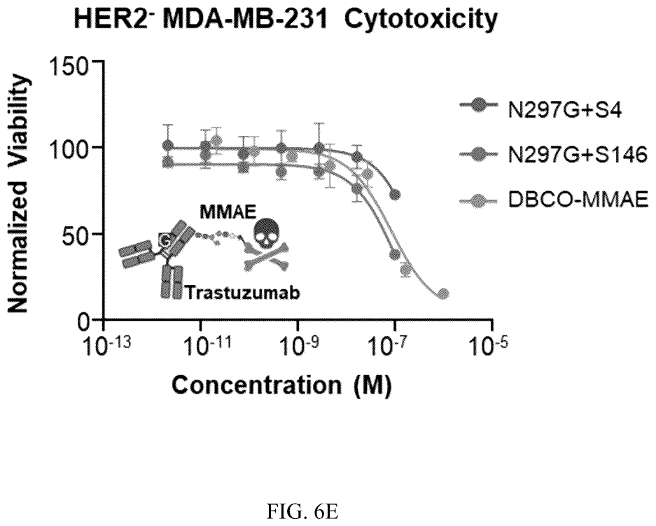

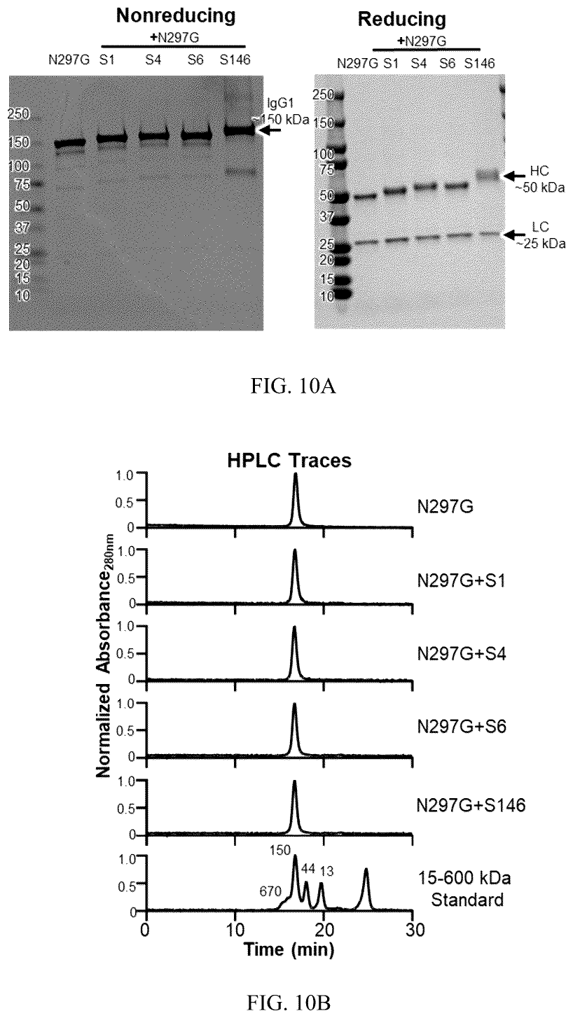

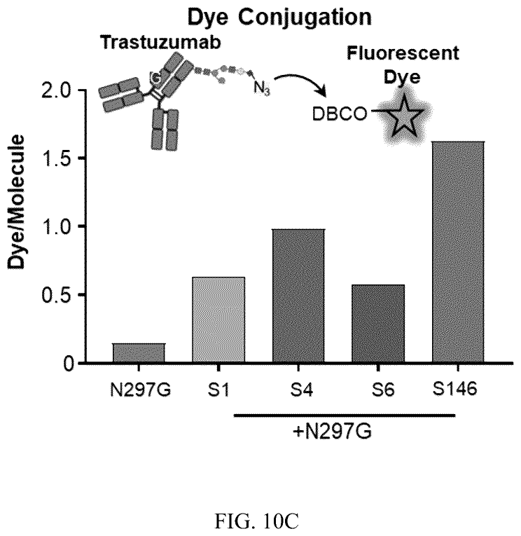

Having established the efficacy of this approach in facilitating fluorescent labeling for applications in research and discovery, it was sought to demonstrate the potential for using this strategy to develop novel biotherapeutics. The FDA-approved anti-human epidermal growth factor receptor 2 (HER2) antibody trastuzumab was selected as a model therapeutic due to its current clinical use in multiple antibody-drug conjugates (Fu et al., Sig Transduct Target Ther 7:1-25 (2022); K. Liu et al., Molecular Cancer 23:62 (2024); and Paul et al., Nat Rev Cancer 24:399-426 (2024)). Due to their minimal effect on FcRn binding, sites S1, S4, and S6 were grafted onto the hIgG1 trastuzumab HC. In this case, the aglycosylated substitution N297G, as opposed to N297A, was used for glycovariants, as it has shown greater promise in terms of downstream developability (Jacobsen et al., J Biol Chem 292:1865-1875 (2017)). Each trastuzumab glycovariant was expressed from HEK-derived Expi293F cells in the presence of 100 μM 1,3,4-O-Bu₃ManNAz, added every 2 days. All of the resulting glycovariants exhibited high purity by SDS-PAGE and analytical high performance liquid chromatography (HPLC), and HC molecular weight shifts were observed compared to the N297G glycomutant, consistent with glycan incorporation (FIGS. 10A and B). Moreover, as observed with F5111 and 1d3, glycomutants for trastuzumab showed identical binding affinity for HER2 compared to the parent antibody (FIG. 6A, Table 3). These glycovariants exhibited similar DBCO dye derivative conjugation compared to the single mutant F5111 and 1d3 glycovariants (FIG. 10C).

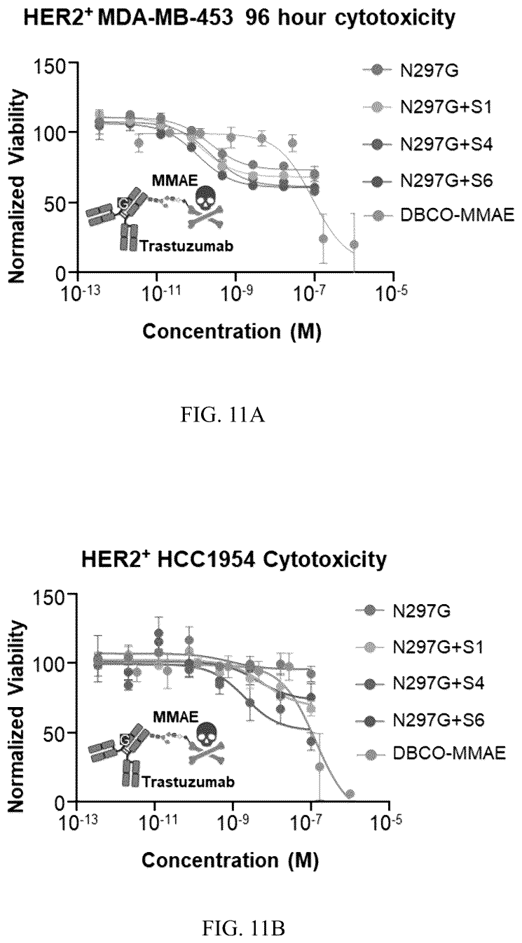

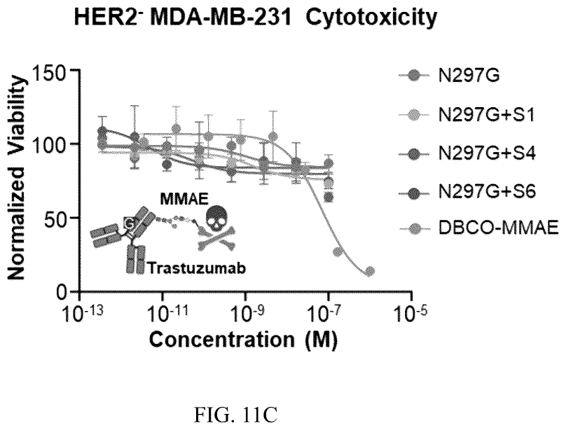

ADCs, which link a disease-targeted antibody to a cytotoxic drug, represent a swiftly growing class of therapeutics, with 13 FDA-approved molecules (including 8 in the last 5 years) and over 100 others in various stages of clinical development (Fu et al., Sig Transduct Target Ther 7:1-25 (2022); K. Liu et al., Molecular Cancer 23:62 (2024); and Paul et al., Nat Rev Cancer 24:399-426 (2024)). To demonstrate that the glycoengineering platform can be used to generate ADCs, trastuzumab glycovariants were linked to the tubulin inhibitor monomethyl auristatin E (MMAE), which is the most commonly employed cytotoxic payload in clinically approved ADCs (Fu et al., Sig Transduct Target Ther 7:1-25 (2022); and K. Liu et al., Molecular Cancer 23:62 (2024)). To this end, the S1, S4, and S6 trastuzumab glycovariants were conjugated to DBCO-PEG4-Val-Cit-MMAE (DBCO-MMAE), an azide-reactive molecule containing a DBCO group, a PEG spacer, a Val-Cit dipeptide, a PAB motif, and an MMAE cytotoxic payload, and the cytotoxic activity of the resulting MMAE-linked anti-HER2 ADCs was evaluated against various human breast cancer cell lines. Each of the trastuzumab glycovariant ADCs exhibited cytotoxicity against the HER2-high cell line SKBR3 with potent (<2 nM) half maximal inhibitory concentration (IC₅₀) (FIG. 6B, Table 6). The unconjugated N297G trastuzumab glycomutant did not induce cytotoxicity, and DBCO-MMAE induced significantly weaker cytotoxicity compared to the ADCs (IC₅₀»100 nM) (FIG. 6B, Table 6), corroborating the targeted killing achieved by the glycovariant ADCs. For the HER2-expressing cell line MDA-MB-453, the unconjugated N297G trastuzumab glycomutant led to very potent cytotoxicity (IC₅₀»230 pM), as previously reported (Rahmati et al., Journal of Applied Biotechnology Reports 7:87-92 (2020)), and the engineered trastuzumab glycovariants ADCs did not further potentiate killing (FIG. 11A, Table 6). The HER2⁺ cell line HCC1954, which has been shown to be less responsive than SKBR3 to MMAE-conjugated ADCs (Kang et al., Nat Biotechnol 37:523-526 (2019)), showed reduced sensitivity to ADC-mediated killing. Notably, in this context, the S4 trastuzumab glycovariant ADC induced more cytotoxicity than the S1 and S6 trastuzumab glycovariant ADCs (FIG. 11B, Table 6), likely due to its higher degree of conjugation (FIG. 10C). Neither the unconjugated N297G trastuzumab glycomutant nor any of the engineered trastuzumab glycovariant ADCs induced cytotoxicity in the HER2− MDA-MB-231 cell line (FIG. 11C), again confirming antigen-dependent cell killing. To augment payload conjugation and thereby enhance the tumor cell killing activity of the engineered ADCs, an ADC was generated using one of the triple mutant glycovariants. The resulting S146 trastuzumab glycovariant expressed with minimal impurity (FIGS. 10A and B), and similar dye conjugation was observed (FIG. 10C) compared to the S146 F5111 glycovariant (FIG. 4C). The S146 trastuzumab glycovariant was formulated as an ADC via DBCO-MMAE conjugation, and the resulting molecule induced potent cell killing (IC₅₀<450 pM, Table 6) of both SKBR3 (FIG. 6C) and HCC1954 (FIG. 6D) cells, with minimal cytotoxicity towards HER2− MDA-MB-231 cells (FIG. 6E). Whereas the triple mutant S146 trastuzumab glycovariant ADC showed only incremental enhancement compared to the already high potency killing by the single mutant S4 trastuzumab glycovariant ADC (FIG. 6C), the S146 trastuzumab glycovariant ADC was nearly 5 times more potent than the S4 trastuzumab glycovariant ADC on HCC1954 cells (FIG. 6D), illustrating the advantage for increased drug conjugation levels in the context of cell lines that are less sensitive to cytotoxic agents. Altogether, ADC design efforts demonstrate that the platform can be used to produce effective and specific molecules that show tumor cell killing potency on par with current state-of-the-art ADCs (K. Liu et al., Molecular Cancer 23:62 (2024) Matsuda & Mendelsohn, Expert Opinion on Biological Therapy 21:963-975 (2021); and Tsuchikama et al., Nat Rev Clin Oncol 21:203-223 (2024) Yamada & Ito, ChemBioChem 20:2729-2737 (2019)).

TABLE 6
Half maximal inhibitory concentrations (IC₅₀) for trastuzumab
glycovariant ADCs against HER2⁺ cell lines.

| | IC₅₀ SKBR3 | IC₅₀ MDA-MB-453 | IC₅₀ HCC1954 |
|---|---|---|---|
| Construct | (nM) (95% CI) | (nM) (95% CI) | (nM) (95% CI) |
| Trastuzumab | n.d. | 0.23 (0.13, 0.39) | n.d. |
| N297G | | | |
| Trastuzumab | 0.79 (0.6, 1.1) | 0.13 (0.11, 0.16) | 6.1 (1.2, 31) |
| N297G + S1 | | | |
| Trastuzumab | 0.65 (0.44, 0.96) | 0.24 (0.15, 0.38) | 2.0 (0.35, 20) |
| N297G + S4 | | | |
| Trastuzumab | 1.7 (0.92, 3.3) | 0.11 (0.087, 0.14) | 4.4 (0.11, 32) |
| N297G + S6 | | | |
| Trastuzumab | 0.17 (0.12, 0.24) | N.A. | 0.45 (0.3, 0.68) |
| N297G + S146 | | | |
| DBCO-PEG4- | 98 (63, 150) | 89 (48, 160) | 131 (74, 240) |
| Val-Cit-PAB- | | | |
| MMAE | | | |

### Engineered Fc Glycovariant Antibody Conjugation to Biomaterials Enables Targeted Gene Delivery

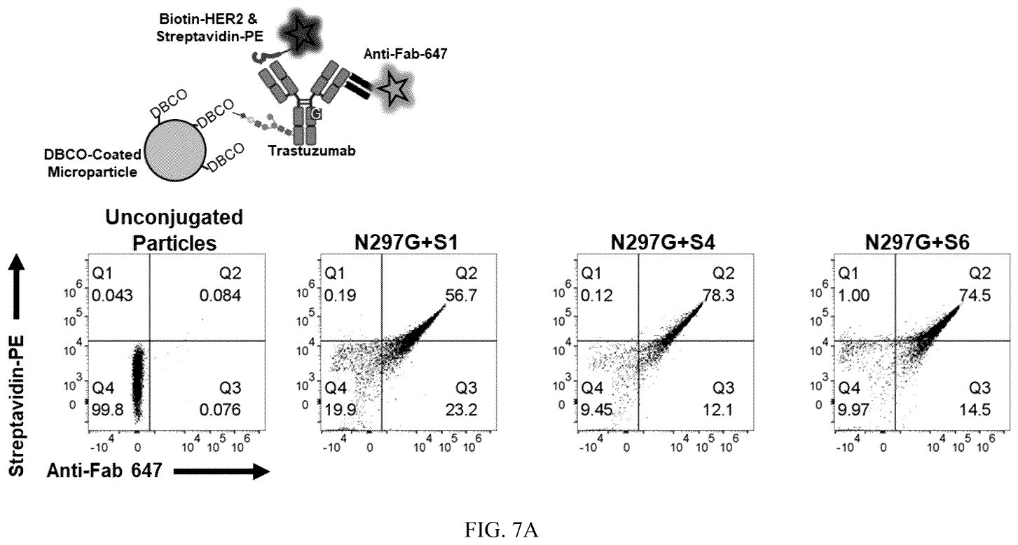

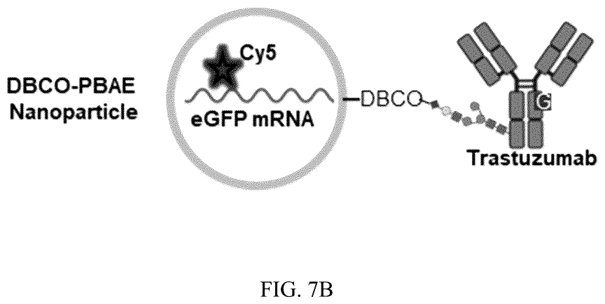

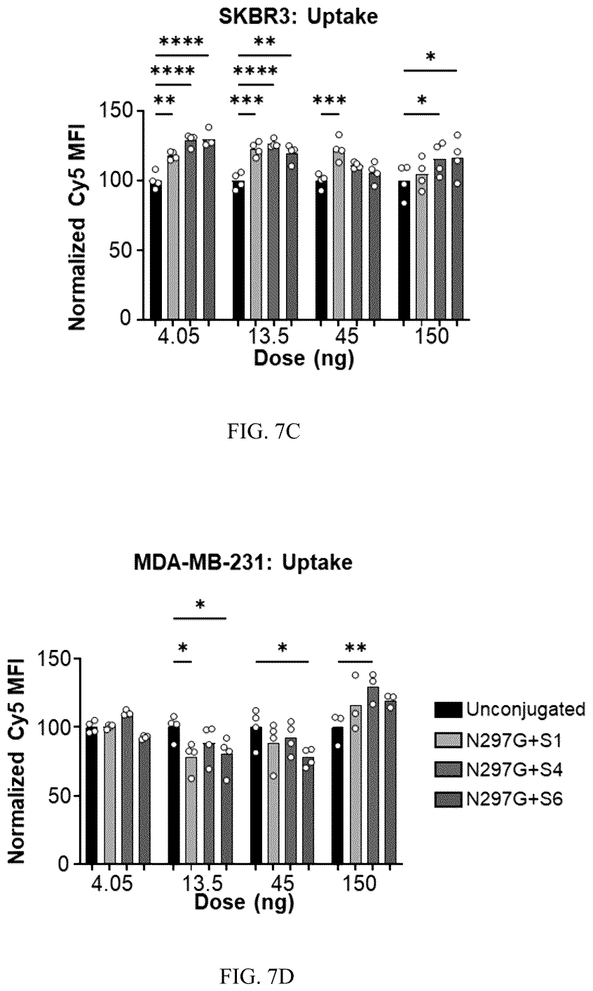

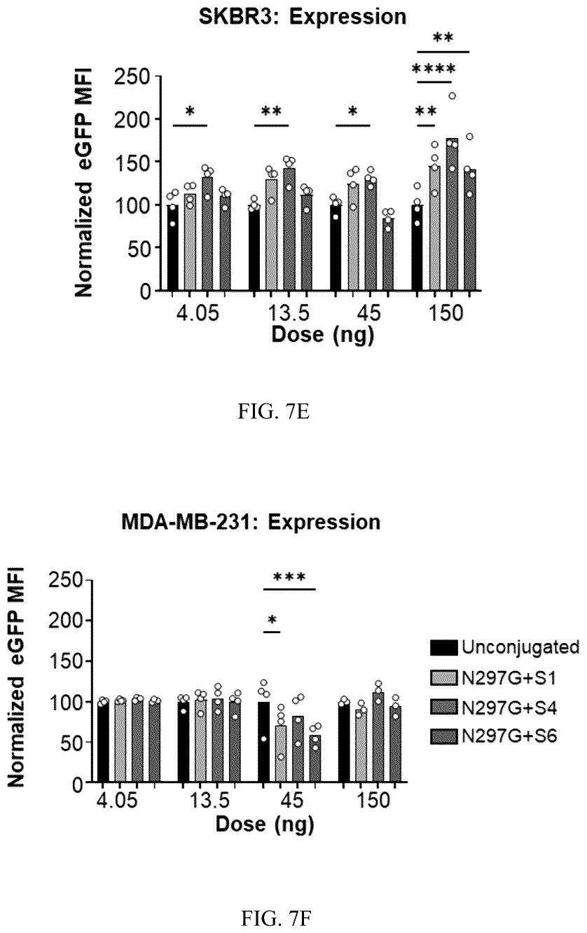

To further elucidate the potential for the glycoengineering strategy in therapeutic applications, it was sought to conjugate the engineered glycovariants to biomaterials scaffolds, specifically polymer-based particles. Such antibody-biomaterials conjugates have vast potential in targeted therapeutic approaches such as biomimetic modulators and gene delivery platforms. As proof of concept, glycoengineered antibodies were conjugated to DBCO-coated magnetic microparticles since the size of these particles allows detection by flow cytometry. It was found that each of the azido-modified S1, S4, and S6 trastuzumab glycovariants was successfully loaded onto DBCO-coated microparticles, while retaining the ability to bind soluble HER2 (FIG. 7A). It was next looked to conjugate the glycovariant antibodies to polymeric nanoparticles as a means of targeting transfection of genetic cargo. Poly(beta-amino ester) (PBAE)-based nanoparticles were employed, which have been used extensively for nucleic acid delivery (Karlsson et al., Expert Opin Drug Deliv 17:1395-1410 (2020)). DBCO-modified PBAE nanoparticles encapsulating a cyanine-5 (Cy5)-labeled mRNA encoding enhanced green fluorescent protein (eGFP) were conjugated to azido-modified S1, S4, and S6 trastuzumab glycovariants, and the resulting nanoparticle conjugates were applied to cells for monitoring of uptake (via Cy5 fluorescence) and gene delivery (via eGFP fluorescence) (FIG. 7B). It was observed that antibody-conjugated nanoparticles containing each of the 3 trastuzumab glycovariants showed increased uptake compared to unconjugated control nanoparticles in HER2-expressing SKBR3 cells (FIG. 7C), whereas antibody-conjugated particles exhibited equivalent or lower uptake compared to unconjugated control nanoparticles in HER2− MDA-MB-231 cells (FIG. 7D). Furthermore, the antibody-conjugated nanoparticles led to higher gene expression of GFP relative to unconjugated control nanoparticles, most prominently at the 150 ng dose, in SKBR3 cells (FIG. 7E), whereas the antibody-conjugated nanoparticles led to lower or equivalent expression relative to unconjugated control nanoparticles at all concentrations with the exception of the 150 ng dose in MDA-MB-231 cells (FIG. 7F). Taken together, these findings establish that the platform is compatible with conjugation to nanoparticles and can be used for targeted gene delivery applications.

### Engineered Fc Glycovariant Antibodies Show Promising Developability Properties

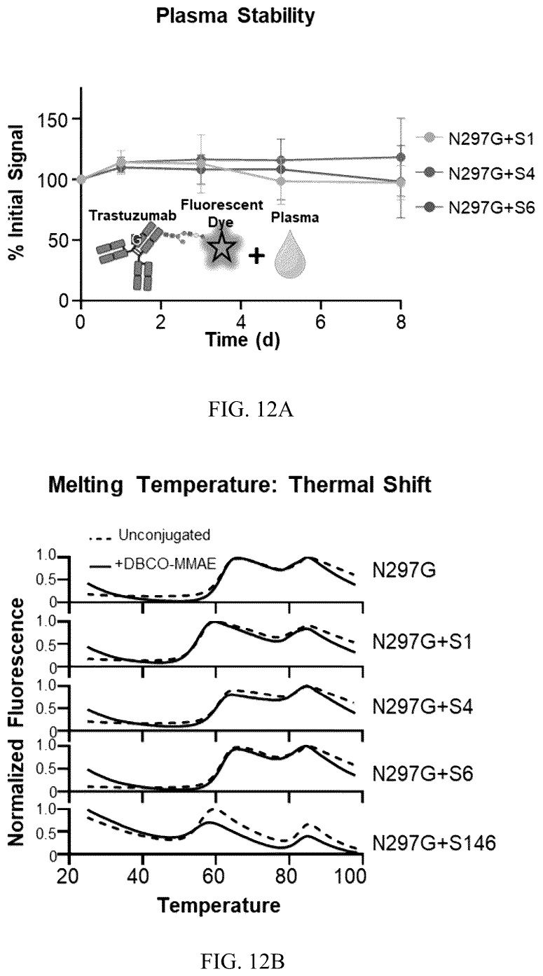

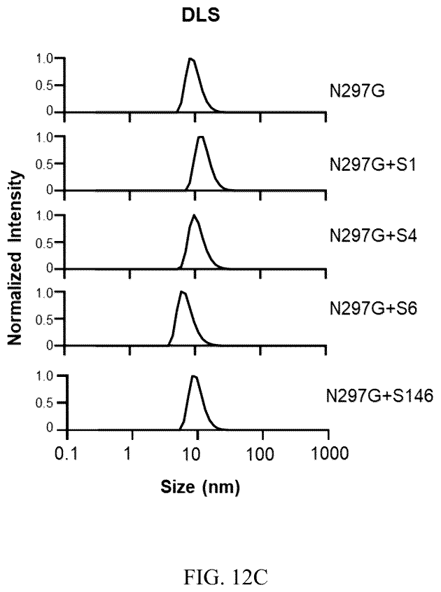

With an eye towards clinical translation of the engineered glycovariant antibodies, several developability properties of the glycovariants and their respective conjugates were thoroughly evaluated. To evaluate the stability of the glycan linkage, stability studies were performed in human plasma using fluorescent dye-conjugated S1, S4, and S6 trastuzumab glycovariants. Over the course of 8 days, decay was not observed in the fluorescent signal (FIG. 12A), indicating that this linkage is stable under physiological conditions. The thermal stability of trastuzumab glycovariants and their respective MMAE-conjugated ADCs was additionally measured. The single mutant S4 and S6 trastuzumab glycovariants had similar melting temperatures to the N297G trastuzumab antibody (FIG. 12B, Table 7). The single mutant S1 trastuzumab glycovariant and the triple mutant S146 glycovariant showed slightly lower melting temperatures, but still exceeded 54° C. Importantly, due to the very mild conditions required for conjugation, the thermal stability was not affected by drug conjugation, as no significant differences in melting temperature were observed between each glycovariant antibodies and its respective ADC (FIG. 12B, Table 7). DLS also indicated that the installed glycans did not induce aggregation in any of the trastuzumab glycovariants that were tested (FIG. 12C). Altogether, these biophysical assays demonstrate that the glycoengineering platform leads to stable attachment of glycans and does not adversely affect antibody developability.

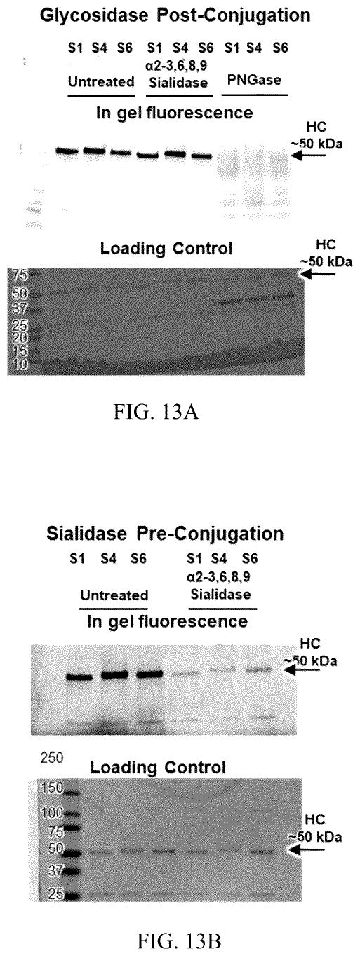

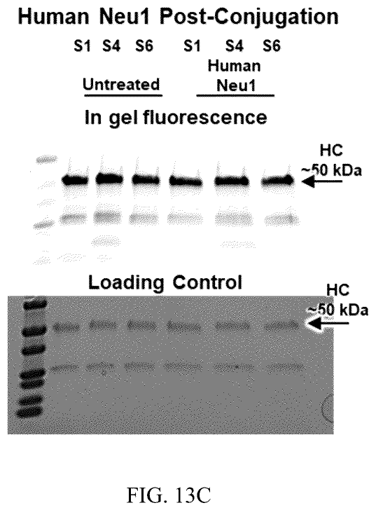

To further characterize the engineered glycovariants, the glycan linkages formed through DBCO-based conjugation were probed by treatment of dye-conjugated S1, S4, and S6 trastuzumab glycovariant antibodies with various glycosidases. As expected, PNGase digestion (which cleaves all N-linked glycans) eliminated all detectable fluorescence for each of the glycovariant antibodies (FIG. 13A). From a translatability standpoint, the ability of circulating sialidases to cut azido-modified sialic acids from glycoconjugates raises the concern that ADCs made using this platform would be unstable in vivo. This concern was allayed, however, by the finding that all 3 dye-conjugated trastuzumab glycovariants were resistant to the α2-3,6,8,9 bacterial sialidase when administered post-conjugation (FIG. 13A). It was suspected that fluorophore linkage may obstruct recognition of the sugar analog by this sialidase. To demonstrate that the incorporated glycans were indeed sialic acids, unconjugated protein was pre-treated with sialidase and then subjected the material to DBCO-fluorophore conjugation. This treatment regimen led to minimal dye conjugation for all 3 trastuzumab glycovariant antibodies (FIG. 13B), demonstrating that the azide groups within the glycovariants were incorporated as sialic acids. Considering the resistance to bacterial sialidase post-conjugation, it was investigated whether these linkages were also resistant to the human sialidase neuraminidase 1, which would be advantageous for plasma stability of the glycan linkage. Encouragingly, it was observed that all 3 dye-conjugated trastuzumab glycovariants were resistant to cleavage by this sialidase as well (FIG. 13C). Overall, it was verified that the newly installed conjugation was indeed site specific, and that the glycan linkages were robust to human sialidase cleavage, potentially boosting plasma stability.

TABLE 7
Melting temperatures for trastuzumab glycovariants
and their respective conjugates.

| Construct | Tm1 | Tm2 |
|---|---|---|
| Trastuzumab N297G | 61.69 | 80.26 |
| Trastuzumab N297G + MMAE | 62.03 | 80.59 |
| Trastuzumab N297G + S1 | 55.94 | 80.26 |
| Trastuzumab | 55.26 | 79.92 |
| N297G + S1 + MMAE | | |
| Trastuzumab N297G + S4 | 61.02 | 81.27 |
| Trastuzumab | 61.02 | 81.94 |
| N297G + S4 + MMAE | | |
| Trastuzumab N297G + S6 | 61.69 | 83.62 |
| Trastuzumab | 62.03 | 81.16 |
| N297G + S6 + MMAE | | |
| Trastuzumab N297G + S146 | 55.74 | 82.96 |
| Trastuzumab | 54.57 | 82.29 |
| N297G + S146 + MMAE | | |

### Methods

### Cytotoxicity Assays

The day before treatment, cells were seeded at 5,000/well in 100 μL complete media in a tissue culture-treated 96-well flat bottom plate (Corning). On the day of experiment, 10 μL of antibody conjugated with monomethyl auristatin E (MMAE) or DBCO-PEG4-Val-Cit-MMAE alone was added in quadruplicate. 96 hours after treatment, cell viability was determined by XTT assay (Thermo Fisher) according to the manufacturer's protocol.

### Microparticle Binding Studies

For conjugation of DBCO magnetic microparticles (VectorLaboratories), 150 μg of particles were incubated with 1 μg of Ab overnight at 4° C. Particles were then washed with PBSA and stained with 50 nM biotinylated HER2 (Sino Biological) for 20 min at room temperature. Particles were then washed with PBSA and stained with a 1:50 dilution of phycoerythrin (PE)-conjugated streptavidin (Thermo Fisher) and a 1:200 dilution of 647-conjugated Alexa Fluor® 647 AffiniPure™ Fab Fragment Goat Anti-Human IgG (H+L) (JacksonImmunoResearch) in PBSA for 20 min on ice. The beads were then washed twice with PBSA and analyzed on a Cytoflex flow cytometer.

### Nanoparticle Preparation and Transfection

A PBAE was synthesized as described previously (Rui et al., Science Advances 8:eabk2855 (2022)). Briefly, bisphenol A glycerolate (1 glycerol/phenol) diacrylate (Sigma-Aldrich, St. Louis, MO) was mixed with 1-dodecylamine (from Alfa Aesar, Tewksbury, MA) and 4-(2-aminoethyl) morpholine (Sigma-Aldrich, St. Louis, MO) (80:20 mol/mol ratio of amines, 2.3:1 mol/mol ratio of vinyl groups to amine) in a 600 mg/mL solution in anhydrous dimethylformamide. The reaction was allowed to proceed with stirring at 85° C. for 48 h. The polymer was diluted to 200 mg/mL in anhydrous tetrahydrofuran and end-capped with diethylentriamine (EMD Millipore, Burlington, MA) at room temperature for with stirring 2 h. The end-capped PBAE was precipitated and washed in diethyl ether, then dried under vacuum at room temperature for 48 h. The purified PBAE was dissolved in anhydrous dimethyl sulfoxide and stored at −20° C.

To measure both uptake and gene expression simultaneously, enhanced green fluorescent protein (eGFP) mRNA (CleanCap, 5-methoxyuridine-modified, Trilink Biotechnologies) was labeled with Cy5 using the Label IT Nucleic Acid Labeling Kit (Mirus Bio, Madison, WI) according to the manufacturer's instructions. The Cy5-labeled eGFP mRNA was mixed with unlabeled eGFP mRNA at a 1:4 ratio for nanoparticle (NP) formulation.

Antibody was conjugated to 1,2-dimyristoyl-rac-glycero-3-methoxypolyethylene glycol-2000-dibenzocyclooctyne (DMG-PEG2k-DBCO) (Nanocs) by incubation overnight at 4° C. with an azide-functionalized antibody. mRNA-loaded PBAE NPs were formed by self-assembly. mRNA was diluted in sodium acetate buffer (NaAc, pH 5), and the PBAE was dissolved in a 90:10 (vol/vol) mixture of ethanol and NaAc. The polymer solution was mixed 1:1 (vol/vol) at a 30:1 mass ratio of polymer to mRNA to form nanoparticles, which were dialyzed against 0.25 mM NaAc using a dialysis cassette with a 50 kDa molecular weight cutoff for 1 h. The salt concentration and pH were raised by addition of 10′ PBS to a final concentration of 0.5′ PBS, and the NPs were allowed to incubate for 30 min before being stabilized by addition of DMG-PEG2k or DMG-PEG-antibody to a final mass ratio of 1:10 DMG-PEG: PBAE. Low-endotoxin sucrose was added to the NPs as a cryoprotectant at a final concentration of 50 mg/mL, and the particles were stored at −80° C. until use. The final NPs contained mRNA at a concentration of 0.02 mg/mL.

Cells were seeded in complete medium 1 day before transfection to allow adhesion (10,000 cells/well in 96-well plates). On the day of transfection, NPs were thawed, diluted in complete medium, and added to cells at a final dosage of 4.05 ng, 13.5 ng, 45 ng, or 150 ng/well in 120 μL volume. Cells were incubated for 24 h with the NPs, then trypsinized and analyzed by flow cytometry (Attune NxT, Thermo Fisher) for Cy5 fluorescence and eGFP expression, using 7-aminoactinomycin D (7-AAD) (Thermo Fisher) to exclude dead cells. Experiments were performed in quadruplicate. Statistical significance was determined by two-way ANOVA with a Dunnett post hoc test.

### Plasma Stability Studies

Plasma stability studies of antibody-fluorophore conjugates were performed as previously described with minor modifications (Dovgan et al., Sci Rep 10:7691 (2020)). For each time point, 500 ng of each BP Fluor 647-labeled antibody was incubated in 66% plasma at 37° C. At each time point, the sample was diluted with SDS sample buffer and snap frozen. The samples were then subjected to SDS-PAGE analysis and in-gel fluorescent imaging. Percent initial signal was calculated by dividing the fluorescent intensity of the conjugate at each time point by the intensity at the starting point. Experiments were performed in duplicate.

### Thermal Stability and Purity Measurements

Antibody melt curves were obtained using a Protein Thermal Shift Kit (Thermo Fisher), following the manufacturer's protocol. Antibody melting temperatures were determined by maxima of the differential melt-curve. For analytical high performance liquid chromatography (HPLC) studies, 10 μg protein was separated on an Acclaim SEC-1000 (7.8×300 mm). Dynamic light scattering measurements were collected using a Zetasizer Pro (Malvern).

### Glycosidase Treatment

For each enzyme, 500 ng of BP Fluor 647 dye-conjugated antibody was digested. PNGase F and α2,3,6,8,9 Neuraminidase (NEB) treatments were performed according to the manufacturer's instructions. Human neuraminidase1 (Prospec) cleavage was performed at a 1:1 mass ratio with antibody in 0.1 M sodium acetate, pH 4.5, as previously described (Guo et al., J. Med. Chem. 61:11261-11279 (2018)). Bands were visualized using in-gel fluorescent imaging on an iBright FL1500 (Thermo Fisher) under the AlexaFluor 647 channel.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A recombinant polypeptide, wherein said recombinant polypeptide contains a fragment crystallizable region (Fc) polypeptide, and wherein said recombinant polypeptide comprises an engineered N-linked glycosylation site.

2. The recombinant polypeptide of claim 1, wherein said Fc polypeptide is a mammalian Fc polypeptide.

3. The recombinant polypeptide of claim 2, wherein said Fc polypeptide is a human Fc polypeptide.

4. The recombinant polypeptide of claim 1, wherein said recombinant polypeptide comprises or consists essentially of an amino acid sequence set forth in any one of SEQ ID NOs:3-8.

5. A nucleic acid construct encoding a recombinant polypeptide of claim 1.

6. An antibody comprising the recombinant polypeptide of claim 1.

7. A method for making an antibody conjugate, said method comprising:
(a) contacting an antibody comprising the recombinant polypeptide of claim 1 with a composition including a carbohydrate comprising a first functional group to incorporate said first functional group into said engineered N-linked glycosylation site of said recombinant polypeptide thereby generating a functionalized antibody; and
(b) contacting said functionalized antibody with a molecule comprising a second functional group to conjugate said molecule to said functionalized antibody; thereby generating said antibody conjugate.

8. The method of claim 7, wherein said carbohydrate is selected from the group consisting of N-acetylmannosamine (ManNAc), N-acetylgalactosamine (GalNAc), N-acetylglucosamine (GlcNAc), fucose, and any analog thereof.

9. The method of claim 7, wherein said first functional group is an azide group and wherein said second functional group is an azide-reactive functional group.

10. The method of claim 9, wherein said carbohydrate comprising said first functional group is a 1,3,4-O-Bu₃ManNAz.

11. The method of claim 9, wherein said azide-reactive functional group is selected from the group consisting of an alkyne group, a dibenzocyclooctyne (DBCO) group, a bicyclononyne (BCN) group, and a triphenylphosphine group.

12. The method of claim 7, wherein said first functional group is a thiol group and wherein said second functional group is a thiol-reactive functional group.

13. The method of claim 12, wherein said thiol-reactive functional group is selected from the group consisting of a maleimide group, acrylamides, vinyl sulfone, acetyl bromide, acetyl iodine, pyridyl disulfide, and tosylated linkers.

14. The method of claim 7, wherein said first functional group is a ketone group and wherein said second functional group is a ketone-reactive functional group.

15. The method of claim 14, wherein said ketone-reactive functional group is selected from the group consisting of a hydrazide group and an aminooxy groups.

16. The method of claim 7, wherein said first functional group is an aldehyde group and wherein said second functional group is an aldehyde-reactive functional group.

17. The method of claim 16, wherein said aldehyde-reactive functional group is selected from the group consisting of a hydrazide group and an aminooxy group.

18. The method of claim 7, wherein said molecule is selected from the group consisting of a small molecule, a nucleic acid, a polypeptide, and a peptide-oligonucleotide conjugate (POC).

19. The method of claim 7, wherein said molecule is selected from the group consisting of an imaging agent and a therapeutic agent.

20. The method of claim 7, wherein said antibody comprising said recombinant polypeptide is produced by a cell in a cell culture, wherein said cell comprises a nucleic acid construct encoding said antibody such that said cell expresses said antibody, and wherein contacting said antibody with said composition comprises supplementing culture media of said cell culture with said composition.


SEQUENCE LISTING (8 sequences)

SEQ ID NO: 1 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 217 aa, Synthetic construct
  VARIANT 67: note = X can be A, G, D, or Q
APELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 217 aa, Synthetic construct
  VARIANT 67: note = X can be N, A, G, D, or Q
APELLGGPSVFLFPPKPKDTLMISRTPEVTCVVVDVSHEDPEVKFNWYVNGTEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 217 aa, Synthetic construct
  VARIANT 67: note = X can be N, A, G, D, or Q
APELLGGPSVFLFPPKPKDTLNISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 217 aa, Synthetic construct
  VARIANT 67: note = X can be N, A, G, D, or Q
APELLGGPSVFLFPPKPKDTLMISRTPEVTCVVVDVSHEDPEVKFNWYVDGVEVHNNKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 217 aa, Synthetic construct
  VARIANT 67: note = X can be N, A, G, D, or Q
APELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVTSCSVMHEALHNHYTQKSLSLSPGK

SEQ ID NO: 7 - protein, 217 aa, Synthetic construct
  VARIANT 67: note = X can be N, A, G, D, or Q
APELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQNGTVFSCSVMHEALHNHYTQKSLSLSPGK

SEQ ID NO: 8 - protein, 217 aa, Synthetic construct
  VARIANT 67: note = X can be N, A, G, D, or Q
APELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMNKTQVSLTCLVKGFYPSDIAVEWESNGQPENNYKTTPPVLDSDGSFFLYSKLTVDKSRWQQGNVFSCSVMHEALHNHYTQKSLSLSPGK